(19) United States
(12) Patent Application Publication
(10) Pub. No.: US 2008/0102476 A1
(43) Pub. Date: May 1, 2008

(54) Analyzing Histamine H4 Receptor-Mediated Effects In Whole Blood

(76) Inventors: Lars Karlsson, San Diego, CA (US); Wai-Ping Leung, San Diego, CA (US); Ping Ling, San Diego, CA (US); Robin Thurmond, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/572,985

(22) PCT Filed: Sep. 24, 2004
(86) PCT No.: PCT/US04/31614

§ 371 Date: Mar. 21, 2006

Related U.S. Application Data
(60) Provisional application No. 60/506,434, filed on Sep. 26, 2003.

(51) Int. Cl. G01N 33/53

(52) U.S. Cl. 435/7.2

(57) ABSTRACT

Assay methods for analyzing whole blood to detect histamine H₄ receptor-mediated effects, such as eosinophil shape change, cytoskeletal change, adhesion molecule up-regulation, or calcium flux are described. Such methods are useful in clinical and diagnostic applications.

### CROSS-REFERENCE TO PRIOR APPLICATION

This application claims the benefit of priority of U.S. Provisional Application No. 60/506,434, filed Sep. 26, 2003.

### FIELD OF THE INVENTION

The invention relates to methods for assaying whole blood to detect or measure histamine H₄ receptor-mediated eosinophil shape change. The invention also relates to assays for detecting or measuring other histamine H₄ receptor-mediated effects in whole blood, such as cytoskeletal changes, adhesion molecule up-regulation, or calcium flux response. Such methods are useful in pharmaceutical research and development, e.g., as biomarker assays to monitor the effects of histamine H₄ receptor modulators administered to patients or subjects in clinical trials.

### BACKGROUND OF THE INVENTION

Certain whole blood assays for investigating eotaxin and other chemokine receptor antagonists have been reported. See, Bryan et al., 2002, “Responses of leukocytes to chemokines in whole blood and their antagonism by novel CC-chemokine receptor 3 antagonists,” Am. J. Respir. Crit. Care Med., 165, 1602-1609. See also, Menzies-Gow et al., 2002, “Eotaxin (CCL11) and eotaxin-2 (CCL24) induce recruitment of eosinophils, basophils, neutrophils, and macrophages as well as features of early- and late-phase allergic reactions following cutaneous injection in human atopic and nonatopic volunteers,” J. Immunol., 169, 2712-2718; Heinemann et al., 2000, “Basophil responses to chemokines are regulated by both sequential and cooperative receptor signaling,” J. Immunol., 165, 7224-7233; Penido et al., 2001, “LPS induces eosinophil migration via CCR3 signaling through a mechanism independent of RANTES and eotaxin,” Am. J. Respir. Cell Mol. Biol., 25, 707-716. There is a need, however, for whole blood assays for histamine H₄ receptor-mediated effects.

Histamine is a biogenic amine playing an important role in the regulation of different physiological systems in the body. Histamine is synthesized from L-histidine by histidine decarboxylation in specific cell types, such as mast cells, basophils, enterochromaffin-like cells and neurons. The diverse biological effects of histamine are mediated through different histamine receptors, which are all G-protein coupled receptors (GPCRs).

Four different histamine receptors, namely the H₁, H₂, H₃, and H₄ receptors, have been identified (Oda et al., 2000, “Molecular cloning and characterization of a novel type of histamine receptor preferentially expressed in leukocytes,” J. Biol. Chem., 275, 36781-36786; Liu et al., 2001, “Cloning and pharmacological characterization of a fourth histamine receptor (H₄) expressed in bone marrow,” Mol. Pharmacol., 59, 420-426; Liu et al., 2001, “Comparison of human, mouse, rat, and guinea pig histamine H₄ receptors reveals substantial pharmacological species variation,” J. Pharmacol. Exp. Ther., 299, 121-130; Morse et al., 2001, “Cloning and characterization of a novel human histamine receptor,” J. Pharmacol. Exp. Ther., 296, 1058-1066; Nguyen et al., 2001, “Discovery of a novel member of the histamine receptor family,” Mol. Pharmacol., 59, 427-433; Zhu et al., 2001, “Cloning, expression, and pharmacological characterization of a novel human histamine receptor,” Mol. Pharmacol., 59, 434-441). The H₄ receptor, a new member of the histamine receptor family identified recently, has low homology with other histamine receptors. Its closest member in the histamine receptor family is the H₃ receptor, which shares only a 35% amino acid homology with the H₄ receptor. Pharmacological properties of the H₄ receptor have been studied using H₄ receptor-transfected cells (Oda et al., 2000, supra; Liu et al., 2001, supra; Morse et al., 2001, supra; Nguyen et al., 2001, supra; Zhu et al., 2001, supra). H₁ and H₂ receptor specific ligands do not bind to the H₄ receptor. In contrast, certain H₃ receptor agonists and antagonists, such as clobenpropit, imetit, R-α-methylhistamine, and thioperamide, show various degrees of cross-reactivity with the H₄ receptor. Recently, antagonists specific for the H₃ or H₄ receptors have been generated. See, e.g., Shah et al., 2002, “Novel human histamine H₃ receptor antagonists,” Bioorg. Med. Chem. Lett., 12, 3309-3312; Jablonowski et al., 2003, “The first potent and selective non-imidazole human histamine H₄ receptor antagonists,” J. Med. Chem., 46(19), 3957-3960; and International Publication No. WO 02/072548. Such compounds can serve as valuable tools for dissecting the biological roles of the H₃ and H₄ receptors.

The four histamine receptors are distinct in their expression profiles and they mediate different biological effects. The H₁ receptor mediates symptoms of allergic reactions, including smooth muscle contractions, vaso-dilation, and sensory nerve activation. The H₂ receptor enhances gastric acid secretion in the stomach. And the H₃ receptor regulates the release of histamine and neurotransmitters by neurons (Hill et al., 1997, “International Union of Pharmacology. XIII. Classification of histamine receptors,” Pharmacol. Rev., 49, 253-278). Expression of H₄ receptors are restricted to cells of the haematopoietic lineage, in particular mast cells, basophils, and eosinophils (Oda et al., 2000, supra; Liu et al., 2001, supra; Morse et al., 2001, supra; Zhu et al., 2001, supra).

The H₄ receptor has been found to mediate mast cell chemotaxis (Hofstra et al., 2003, “Histamine H₄ receptor mediates chemotaxis and calcium mobilization of mast cells,” J. Pharmacol. Exp. Ther., 305, 1212-1221). The chemotactic effects of histamine on eosinophils have also been suggested in early studies (Clark et al., 1975, “The selective eosinophil chemotactic activity of histamine,” J. Exp. Med., 142, 1462-1476). Recently, eosinophil chemotaxis toward histamine was found to be blocked by thioperamide and was therefore suggested to be mediated by the H₄ receptor (O'Reilly et al., 2002, “Identification of a histamine H₄ receptor on human eosinophils-role in eosinophil chemotaxis,” J. Recept. Signal Transduct. Res., 22, 431-448).

Eosinophils are bone marrow-derived granulocytic leukocytes that normally reside in tissues, especially in the respiratory and intestinal systems and in the uterus. Eosinophil numbers in the blood stream are relatively low and the control of eosinophil migration toward tissues has been attributed to adhesion molecules and chemokines (Lukacs, 2001, “Role of chemokines in the pathogenesis of asthma,” Nat. Rev. Immunol., 1, 108-116; Tachimoto et al., 2002, “Cross-talk between integrins and chemokines that influences eosinophil adhesion and migration,” Int. Arch. Allergy Immunol., 128, 18-20). Eosinophils are important effector cells in the late-phase allergic response and they have been implicated in the pathogenesis of allergic diseases (Bousquet et al., 1990, “Eosinophilic inflammation in asthma,” N. Engl. J. Med., 323, 1033-1039). Activation of eosinophils results in the release of toxic granule proteins that are thought to cause airway epithelial damage and the development of bronchial hyper-reactivity in asthma.

### SUMMARY OF THE INVENTION

Having established that the histamine H₄ receptor mediates eosinophil chemotaxis with cell shape change and adhesion molecule up-regulation as discussed below, whole-blood histamine assays have now been developed.

Thus, in one general aspect, the invention relates to a method for assaying for a histamine H₄ receptor-mediated effect, comprising performing an investigative assay on a treated sample of whole blood by steps comprising: treating whole blood from a source with a histamine H₄ receptor antagonist, yielding the treated sample; adding to the treated sample an assay reagent selected from histamine and specific histamine H₄ receptor agonists, yielding an assay sample; and analyzing the assay sample to detect the histamine H₄ receptor-mediated effect. In another general aspect, the invention relates to a method for assaying for a histamine H₄ receptor-mediated effect, comprising performing a control assay on a sample of whole blood untreated with any histamine H₄ receptor antagonist by steps comprising: adding to the untreated sample an assay reagent selected from histamine and specific histamine H₄ receptor agonists, yielding a control sample; and analyzing the control sample to detect the histamine H₄ receptor-mediated effect. In preferred embodiments, the control assay and the investigative assay are combined for comparative purposes.

Advantages of the inventive assays, and illustrative embodiments and alternative features, will be apparent from the detailed description below taken in conjunction with the appended figures.

### BRIEF DESCRIPTION OF THE DRAWING FIGURES

FIGS. 1A and 1B depict results illustrating that H₄ receptor expression is restricted to eosinophils and dendritic cells. For FIGS. 1A and 1B, human SK-N-MC cells transfected with H₁, H₂, H₃ or H₄ receptor were used as controls for specificity of histamine receptor detection. G3PDH mRNA in RNA samples was amplified with specific primers as controls in PCR reactions.

FIG. 1A: RT-PCR detection of H₄ receptor mRNA in different purified cell types and cell lines. Total RNA from different cell types were reversed transcribed and used as templates for PCR (25 PCR cycles were performed for amplification of H₄ receptor).

FIG. 1B: Human eosinophils express H₄ but not H₃ receptors. H₃ or H₄ receptor mRNA in human eosinophils was detected by RT-PCR using specific primers. Twenty-five PCR cycles were performed for amplification of H₄ receptor.

FIGS. 2A-2E illustrate results showing that histamine induces eosinophil shape change.

FIG. 2A: Flow cytometry analysis illustrating that eosinophils (R1) are distinguished from neutrophils (R2) in human PMNL by gating on cells with high levels of autofluorescence.

FIG. 2B: Flow cytometry analysis illustrating that the majority of the cell population with high autofluorescence, gated as R1 group, is CCR3⁺ eosinophils.

FIG. 2C: Flow cytometry analysis illustrating that the majority of the cell population with low autofluorescence, gated as R2 group, is CD16⁺ neutrophils.

FIG. 2D: Histamine-induced cell shape change on eosinophils. Human PMNL were treated with 1 μM histamine for 10 minutes and the change in cell shape was monitored by flow cytometry. Human eosinophils were gated in flow cytometry analysis based on their autofluorescence as distinguished from the autofluorescence of neutrophils (Fee FIGS. 2A and 2B. The cell size in histamine-treated samples was compared to that of the untreated control samples. The means of cell size in forward scattered signal (FSC) are shown.

FIG. 2E: No histamine-induced cell shape change on neutrophils. Human PMNL were treated with 1 μM histamine for 10 minutes and the change in cell shape was monitored by flow cytometry. Human neutrophils were gated in flow cytometry analysis based on their autofluorescence as distinguished from the autofluorescence of eosinophils (Fee FIGS. 2A and 2C. The cell size in histamine-treated samples was compared to that of the untreated control samples. The means of cell size in forward scattered signal (FSC) are shown.

FIGS. 3A-3E depict results showing the kinetics and potency of histamine in triggering eosinophil shape change.

FIG. 3A: Results demonstrate the kinetics of histamine-induced eosinophil shape change. Human PMNL were treated with 1 M histamine and the change in eosinophil cell shape at different time points was studied by flow cytometry. The percentage of cell shape change was calculated based on the increase from those of untreated samples. Data are mean±SD, n=3.

FIG. 3B: The shape change response of human eosinophils to histamine under different conditions was studied. Data are mean±SD, n=3.

FIG. 3C: Results of titration of histamine effects on human eosinophil shape change and comparison with chemokines are illustrated. Human PMNL were treated with different concentrations of histamine or chemokines (eotaxin-2 or MCP-3) for 10 min. Eosinophil shape change was monitored by flow cytometry. Data are mean±SD, n=2.

FIG. 3D: Results of determination of EC₅₀ values of histamine and chemokines on eosinophil shape change are illustrated. Human PMNL were treated with different concentrations of histamine or chemokines (eotaxin, eotaxin-2, and MCP-3). Eosinophil shape change was monitored by flow cytometry. Data shown is a representative of 5 repeated experiments. EC₅₀ values were calculated with the GraphPad Prism program.

FIG. 3E: Results are illustrated showing that histamine enhances eosinophil shape change when combined with chemokines MCP-3. Human PMNL were treated with histamine in combination of chemokine MCP-3 for 10 minutes. Eosinophil shape change was monitored by flow cytometry. Data shown is a representative of 4 repeated experiments.

FIGS. 4A-4C depicts results showing that histamine-induced eosinophil shape change is mediated by the H₄ receptor.

FIG. 4A: Histamine induced eosinophil shape change was blocked by the H₄ receptor antagonist (5-chloro-1H-indol-2-yl)-(4-methyl-piperazin-1-yl)-methanone (Compound A), the H₃/H₄ receptor antagonist thioperamide, but not by H₁, H₂, or H₃ receptor antagonists. The H₁, H₂ and H₃ receptor antagonists used in studies were diphenhydramine, ranitidine, and (7-methyl-2-[4-(3-piperidin-1-yl-propoxy)-phenyl]-imidazo[1,2-a]pyridine) (Compound B), respectively. Human PMNL were pre-treated with 10 μM of different histamine receptor antagonists, followed by 10-min treatment with 1 μM histamine. Eosinophil shape change was monitored by flow cytometry. The percentage of cell shape change was calculated based on the increase from those of untreated samples. Data shown is a representative of three repeated experiments. Data are mean±SD and n=3. Statistical significance (P value) of the differences between samples and histamine only control were determined by the Student T-test.

FIG. 4B: Determination of IC₅₀ values of Compound A, thioperamide, and H₃ receptor antagonist (Compound B) on histamine-induced eosinophil shape change. Human PMNL was pre-treated with different antagonists for 10 minutes (min) before inducing cell shape change with 1 μM histamine. Eosinophil shape change was monitored by flow cytometry. The percentage of inhibition was calculated based on the decrease in shape change compared to samples treated with 1 μM histamine only. Data shown is a representative of four repeated experiments. IC₅₀ values were calculated with the GraphPad Prizm program.

FIG. 4C: Concentration-dependent effects of histamine, H₃/H₄ receptor agonist imetit, and H₄ receptor antagonist clobenpropit on human eosinophil shape change.

FIGS. 5A-5C depicts results demonstrating that histamine-induced adhesion molecule expression on eosinophils is mediated by the H₄ receptor.

FIG. 5A: Cell surface expression of adhesion molecules CD11b/CD18 and CD54 on eosinophils was up-regulated by histamine. Human PMNL were treated with different concentrations of histamine or chemokine eotaxin-2 for 10 min at 37° C. Cell samples were fixed with paraformaldehyde and stained with FITC-conjugated antibodies specific for CD11b, CD11a or CD54. Expression of adhesion molecules on eosinophils was monitored by flow cytometry. The percentage of up-regulation was calculated based on the increase from the expression levels of untreated samples.

FIG. 5B: Kinetics of adhesion molecule up-regulation on eosinophils. Human PMNL were treated with 1 μM histamine or 1 nM chemokine eotaxin-2 for 10 min or 60 min at 37° C. Cell samples were fixed with paraformaldehyde and stained with FITC-conjugated antibodies specific for CD11b or CD54. Data are mean±SD and n=3.

FIG. 5C: Histamine-induced adhesion molecule up-regulation on eosinophils was blocked by the H₄ receptor antagonist Compound A and the H₃/H₄ receptor antagonist thioperamide, but not by H₁, H₂ or H₃ receptor antagonists. The H₁, H₂ and H₃ receptor antagonists used in studies were diphenhydramine, ranitidine, and Compound B, respectively. Human PMNL were pre-treated with 10 μM of different histamine receptor antagonists for 10 min, followed by 10-min treatment with 1 M histamine at 37° C. Cell samples were fixed with paraformaldehyde and stained with FITC-conjugated antibodies specific for CD11b or CD54. Data are mean±SD and n=3. Statistical significance (P value) of the differences between samples and histamine only control were determined by the Student T-test.

FIGS. 6A-6E depicts results showing that histamine-induced human eosinophil chemotaxis is mediated by the H₄ receptor.

FIG. 6A: Titration of histamine effects on human eosinophil chemotaxis. Chemotaxis of purified human eosinophils toward different concentration of histamine was studied in a Transwell system. Human eosinophils were placed in the transwell and histamine was added in the lower chamber. Eosinophils migrated into the lower chambers after 2 hours (h) incubation were counted for 1 min by flow cytometry. Data shown is a representative of three repeated experiments. Data are mean±SD and n=3. Statistical significance (P value) of the difference between sample and untreated control was determined by the Student T-test.

FIG. 6B: Determination of EC₅₀ values of histamine and chemokines on eosinophil chemotaxis. Human eosinophil chemotaxis was studied with a titration of histamine or chemokines eotaxin-2 or MCP-3. Data shown is a representative of two repeated experiments. Data are mean±SD and n=3. EC₅₀ values were calculated with the GraphPad Prizm program.

FIG. 6C: Histamine enhanced chemokine-induced eosinophil chemotaxis. The effects of histamine (0.5 μM) on eosinophil chemotaxis induced by different concentrations of chemokine eotaxin-2 or MCP-3 were studied. Data shown is a representative from three repeated experiments. Data are mean±SD and n=3. Statistical significance (P value) was determined by the Student T-test.

FIG. 6D: Histamine-induced eosinophil chemotaxis was blocked by the H₄ receptor antagonist Compound A and the H₃/H₄ receptor antagonist thioperamide, but not by H₁, H₂ or H₃ receptor antagonists. The H₁, H₂ and H₃ receptor antagonists used in studies were diphenhydramine, ranitidine, and Compound B, respectively. Ten μM histamine was added in the lower chamber while 10 μM of different histamine receptor antagonists was added in both chambers. Data shown is representative of four repeated experiments. Data are mean±SD and n=3. Statistical significance (P value) was determined by the Student T-test.

FIG. 6E: Determination of IC₅₀ values of H₄ receptor antagonists Compound A and thioperamide in eosinophil chemotaxis assays. One μM histamine was added in the lower chamber while different concentrations of Compound A or thioperamide were added in both chambers. The percentage of inhibition was calculated based on the decrease in migrated cell numbers compared to samples treated with 1 μM of histamine only. Data shown is a representative from four repeated experiments. Data are mean±SD and n=3.

FIG. 7 depicts results showing that histamine and eotaxin2 induce human eosinophil shape change in a whole blood GAFS assay.

FIG. 8 depicts results demonstrating that a histamine H₄ receptor (H4R) antagonist blocks histamine-induced shape change.

FIGS. 9A and 9B depict results of whole blood shape-change assays showing that two commercially available H4R agonists, imetit and clobenpropit, mimic histamine in inducing eosinophil shape change.

FIG. 10 depicts the resolving of an H4-specific shape-change response by treatment of whole blood samples with both histamine and the H2 antagonist, ranitidine.

FIG. 11 shows that increasing concentrations of Compound A inhibit, in a dose-dependant fashion, the H4-specific shape change response that is resolved by co-treatment of whole blood samples with both histamine and ranitidine.

The experimental results depicted in the figures are further discussed in the following detailed description, which discusses illustrative and preferred embodiments of the inventive assays as well as background experiments.

### DETAILED DESCRIPTION OF THE INVENTION AND ITS PREFERRED EMBODIMENTS

Since mast cells are the major producers of histamine and both mast cells and eosinophils are known effector cells in allergic reactions, the role of mast cells in the recruitment of eosinophils via histamine was investigated by performing experiments described below. As discussed below, eosinophils respond to histamine in changing cell shape, up-regulation of adhesion molecules, and chemotaxis. Using H₄ receptor-specific antagonists, we determined that all these responses are mediated by the H₄ receptor expressed on eosinophils.

### Background Experiments

### Materials.

Human cell lines HMC-1, HL60.15, and primary HUVEC cells were purchased from American Type Culture Collection (Rockville, Md.). RNeasy kit was from Qiagen (Valencia, Calif.). RT reaction kits and ExpressHyb solution were from Invitrogen (Carlsbad, Calif.). The H₃ receptor specific antagonist Compound B as well as the H₄ receptor specific antagonist Compound A were synthesized (see WO 01/74815 and WO 02/072548, the disclosures of which are incorporated by reference herein) and their histamine receptor specificities were described previously (Shah et al., 2002, supra; Jablonowski et al., 2003, supra). All chemokines and cytokines were purchased from R&D Systems (Minneapolis, Minn.). All antibodies were purchased from BD PharMingen (San Diego, Calif.). All other reagents were purchased from Sigma (St. Louis, Mo.). Histamine, imetit, clobenpropit, and all other reagents were purchased from Sigma (St. Louis, Mo.).

### Purification of Different Human Haematopoietic Cell Types.

Eosinophils and neutrophils were purified from blood samples collected from healthy volunteers. Briefly, platelet-rich plasma was removed by centrifugation of heparinized whole blood. Polymorphonuclear leukocytes (PMNL), which are enriched with neutrophils and eosinophils, were separated from peripheral blood mononuclear cells (PBMC) by centrifugation at 2000 rpm for 20 min over a discontinuous plasma-Percoll gradient (density 1.082 g/ml). Red blood cells in PMNL were removed by hypotonic shock lysis. PMNL were stained with Hematoxylin and Eosin (H&E) and a differential cell count was performed. Eosinophil counts ranged from 2-10% of the total PMNL number. Human neutrophils were purified from PMNL by positive selection using anti-CD16 conjugated micro-beads in a magnetic cell separation system (AutoMACS from Miltenyi Biotec, Auburn, Calif.). PMNL were incubated with anti-CD16 conjugated micro-beads in PBS containing 0.5% BSA and 2 mM EDTA, which selectively bind to neutrophils. Neutrophils were purified by passage of the cell suspensions through a magnetic field in the AutoMACS system. Eosinophils were purified from the PMNL by negative selection. PMNL were incubated with a cocktail of anti-CD16, anti-CD3, anti-CD19 and anti-CD14 conjugated micro-beads in PBS containing 0.5% BSA and 2 mM EDTA, which selectively bind to neutrophils, T cells, B cells and monocytes, respectively in the PMNL suspension. Eosinophils were purified by removing cells bound to micro-beads in the AutoMACS system, resulting in eosinophil populations of >97.5% purity according to H&E stain. Purified eosinophils were washed once in buffer (PBS containing 10 mM Ca²⁺ and Mg²⁺, 10 mM HEPES, 10 mM glucose, and 0.1% BSA, pH 7.2-7.4) and used immediately for experiments.

Human CD4⁺ T cells were purified from PBMC. Human PBMC were separated from PMNL over a discontinuous plasma-Percoll gradient (density 1.082 g/ml). CD4⁺ T cells in PBMC were purified by positive selection using anti-CD4 conjugated micro-beads in AutoMACS system. CD4⁺ T cells were stimulated for 7 days with immobilized anti-CD3 (culture plates were coated with 5 μg/ml of anti-CD3 in PBS overnight and then rinsed twice with PBS before use) and 2 μg/ml soluble anti-CD28 in the presence of cytokines or antibodies for T cell differentiation. For type I helper T cell differentiation, 10 ng/ml human IL-12 and 10 μg/ml anti-IL-4 were added in culture medium. For type II helper T cell differentiation, 10 ng/ml human IL-4 and 10 μg/ml of anti-IL-12 and anti-IFN-γ were added in culture medium. Human IL-2 was added in cultures at 20 U/ml on day 4 of T cell stimulation. T cells on day 7 were collected for RNA preparation. Type I or type II helper T cells were characterized by their production of IFN-γ or IL-4, respectively. To confirm the effector cell types after the 7 day differentiation, aliquots of T cells were re-stimulated overnight with immobilized anti-CD3 and culture supernatants were tested for IL4 or IFN-γ by ELISA.

CD8⁺ T cells in PBMC were purified by positive selection using anti-CD8 conjugated micro-beads in AutoMACS system. Purified CD8⁺ T cells were stimulated overnight with immobilized anti-CD3 and anti-CD28 in RPMI 1640 medium. Cells after overnight activation were harvested for RNA preparation.

Monocytes in PBMC were purified by positive selection using anti-CD14 conjugated micro-beads in AutoMACS system. Dendritic cells were generated from blood monocytes by culturing purified monocytes for 10 days in the presence of 500 U/ml IL4 and 800 U/ml GM-CSF to reach the immature dendritic cell phenotype. Immature dendritic cells were then treated with 100 U/ml TNF-α for 24 h to drive the cells to the mature dendritic cells phenotype. Mature dendritic cells were used for RNA preparation.

### Differentiation of Human Eosinophilic Cell Line.

Human HL60.15 cell line was cultured in RPMI 1640 medium containing 10% FCS and differentiated into eosinophils by treating cells with 0.5 μM butyric acid and 10 ng/ml IL-5 for 2 days.

### Detection of H₄ and H₃ Receptor RNA Expression.

Total RNA was extracted from purified human cells using the RNeasy kit (Qiagen) and reverse transcribed to cDNA using the RT reaction kit (Invitrogen). H₄ receptor RNA was detected by RT-PCR using human H₄ receptor specific primers 5′-ATGCCAGATACTAATAGCACA and 5′-CAGTCGGTCAGTATCTTCT. The amplified PCR band for H₄ receptor is 1170 bp. H₃ receptor RNA was detected by using human H₃ receptor specific primers 5′-ATGGAGCGCGCGCCGCCCGACGGG and 5′-ATGAAGAAGAAAACATGTCTG. The amplified PCR band for H₃ receptor is 1120 bp.

### Measurement of Eosinophil Shape Change using Flow Cytometry.

Human PMNL samples were used to study eosinophil shape change response. PMNL were prepared as described above and cells were resuspended in assay buffer (PBS containing 10 mM Ca²⁺ and Mg²⁺, 10 mM HEPES, 10 mM glucose, and 0.1% BSA, pH 7.2-7.4). Aliquots of cells (5×10⁵ PMNL in 80 μl assay buffer) were pretreated with histamine receptor analogues (diphenhydramine, ranitidine, thioperamide, Compound A or H₃ receptor antagonist) for 10 min at room temperature before addition of histamine or chemokines in 1.2-ml polypropylene cluster tubes (Costar, Cambridge, Mass.) in a final volume of 100 μl. The tubes were placed in a 37° C. water bath for 10 min (or as indicated), after which they were transferred to an ice-water bath, and 250 μl of ice-cold fixative (2% paraformaldehyde in PBS) was added to terminate the reaction and to maintain the cell shape change. The cell shape change was analyzed with the flow cytometer (Becton Dickinson, Mountain View, Calif.). Eosinophils in PMNL were gated based on their high autofluorescence relative to that of neutrophils. Cell shape change was monitored in forward scatter signals. To identify eosinophils and neutrophils in PMNL, cells were stained on ice for 30 min with saturating concentrations of FITC-conjugated anti-CCR3 or anti-CD16 antibodies, which are specific for eosinophils or neutrophils, respectively. Samples after antibody staining were analyzed in flow cytometry.

### Detection of Cell Surface Expression of Adhesion Molecules.

Purified eosinophils were used to study cell surface expression of adhesion molecules. Eosinophils were resuspended in PBS containing 10 mM Ca²⁺ and Mg²⁺, 10 mM HEPES, 10 mM glucose, and 0.1% BSA, pH 7.2-7.4. Aliquots of cells (5×10⁵ PMNL) were pretreated with histamine receptor analogues (as described above) for 10 min before addition of histamine or chemokines in 1.2-ml polypropylene cluster tubes (Costar, Cambridge, Mass.) in a final volume of 100 μl. The tubes were placed in a 37° C. water bath for 10 min (or as indicated), after which they were transferred to an ice-water bath, and 250 μl of ice-cold fixative (0.5% paraformaldehyde in PBS) was added. Samples were then incubated on ice for 30 min with saturating concentration of either FITC or phycoerytherine-conjugated anti-CD11a, anti-CD11b or anti-CD54 antibodies, washed, and then analyzed by flow cytometry.

### In vitro Chemotaxis Assay.

Transwells (Costar, Cambridge, Mass.) with 5 μm pore size were coated with 100 μl of 100 ng/ml human fibronectin (Sigma) for 2 h at room temperature. After removal of excess fibronectin, 600 μl of RPMI-1640 medium containing 0.5% BSA and different concentrations of histamine (0.01-100 μM) was added to the bottom chamber, Eosinophils (2×10⁵/well) were added to the top chamber. Histamine receptor analogues (diphenhydramine, ranitidine, thioperamide, Compound A or Compound B) were added to both the top and bottom chambers to a final concentration of 10 μM or at other concentration as stated in figure legends. The plates were incubated for 2 h at 37° C. and the number of cells migrated to the bottom chamber was counted for 1 min using flow cytometer.

### Statistics.

Experimental data are presented as mean±standard deviation (SD) from the number (n) of independent samples. The IC₅₀ or EC₅₀ values were calculated from the concentration-effect curves by non-linear regression analysis using GraphPad Prizm (GraphPad Software Inc., Philadelphia, USA). Statistical significance (P) was determined by the Student T-test.

### Results: Eosinophils express H₄ Receptors but not H₃ Receptors.

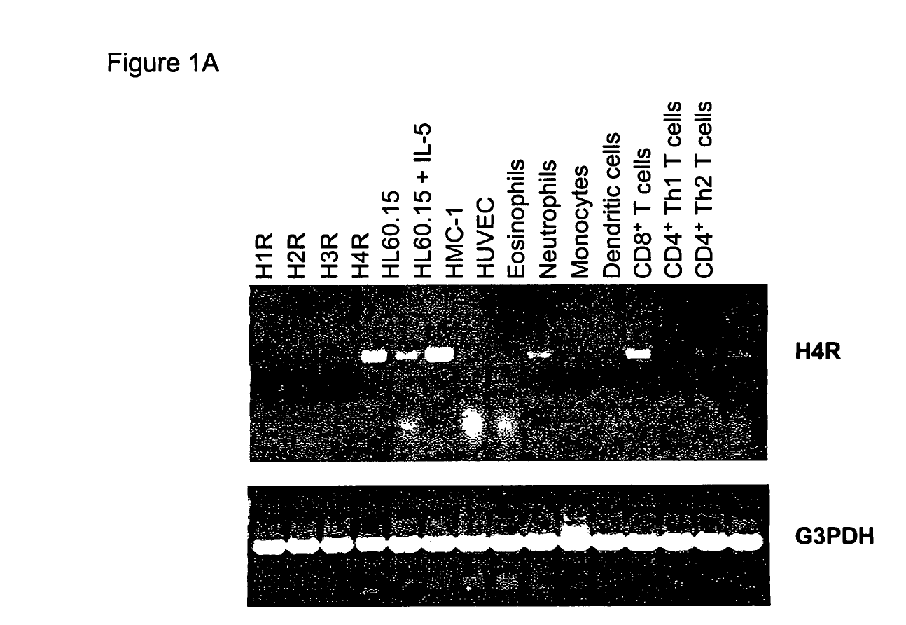

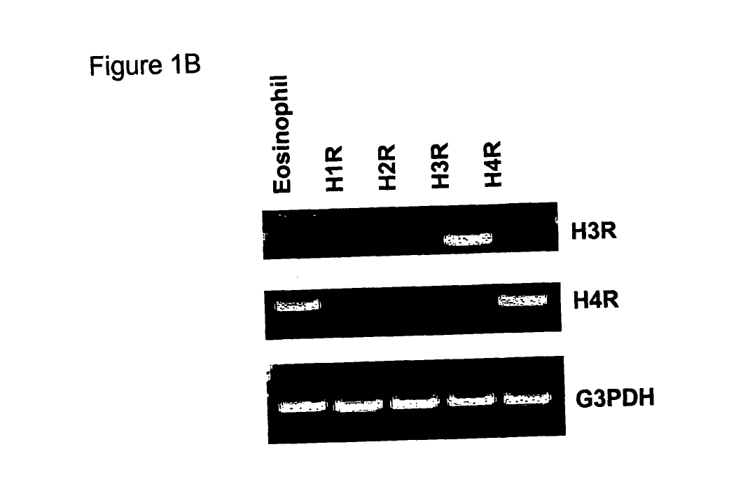

Expression of H₄ receptors in different purified human haematopoietic cell types and cell lines was studied. Significant levels of H₄ receptor mRNA were detected in eosinophils and dendritic cells by RT-PCR (FIG. 1A). In contrast to the H₄ receptor expression in cosinophils, H₃ receptors were not detected in these cells (FIG. 1B). Minute expression of H₄ receptor was found in CD₄₊ Th1 and Th2 effector cells, but was not detected in neutrophils, monocytes and activated CD8⁺ T cells (FIG. 1A). H₄ receptor expression was also detected in eosinophilic precursor cell line HL60.15 and its expression was significantly increased when cells were induced by IL-5 to differentiate into eosinophils. The human HMC-1 mast cell line expressed a detectable level of H₄ receptors.

### Results: H₄ Receptors Mediate Eosinophil Shape Change.

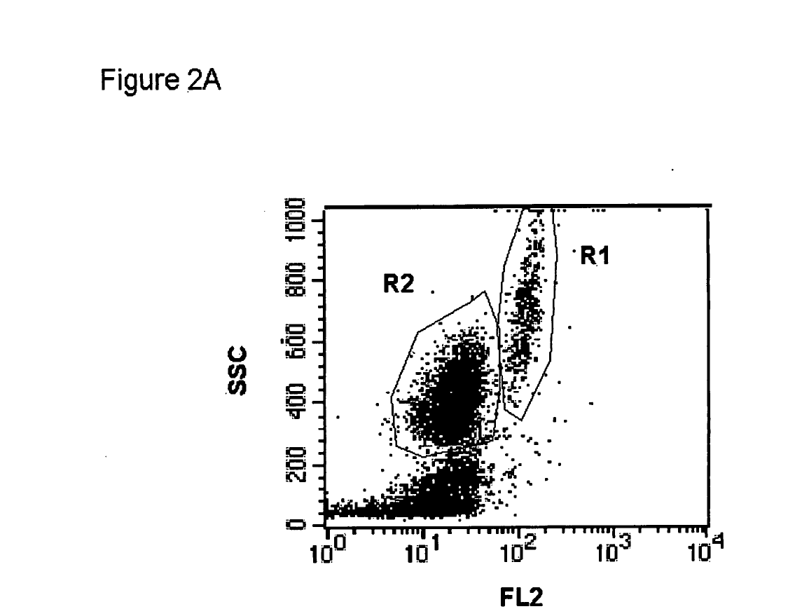

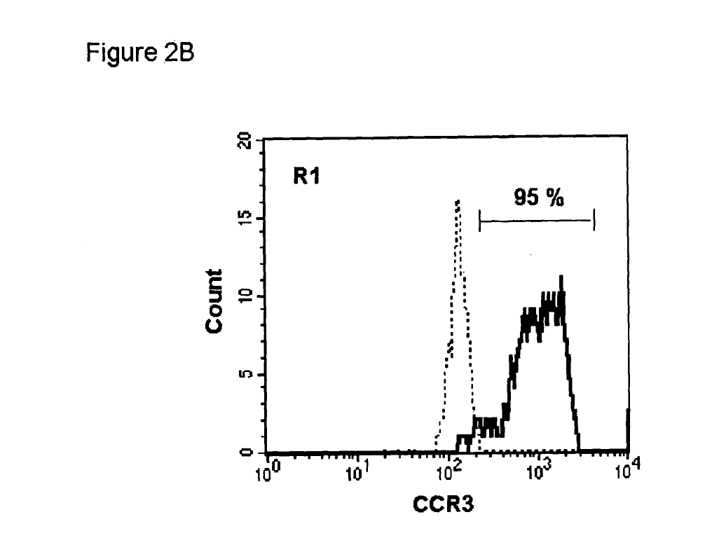

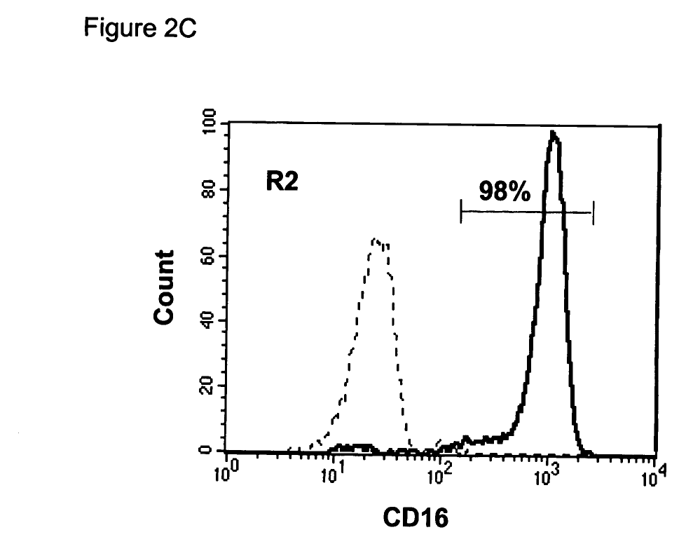

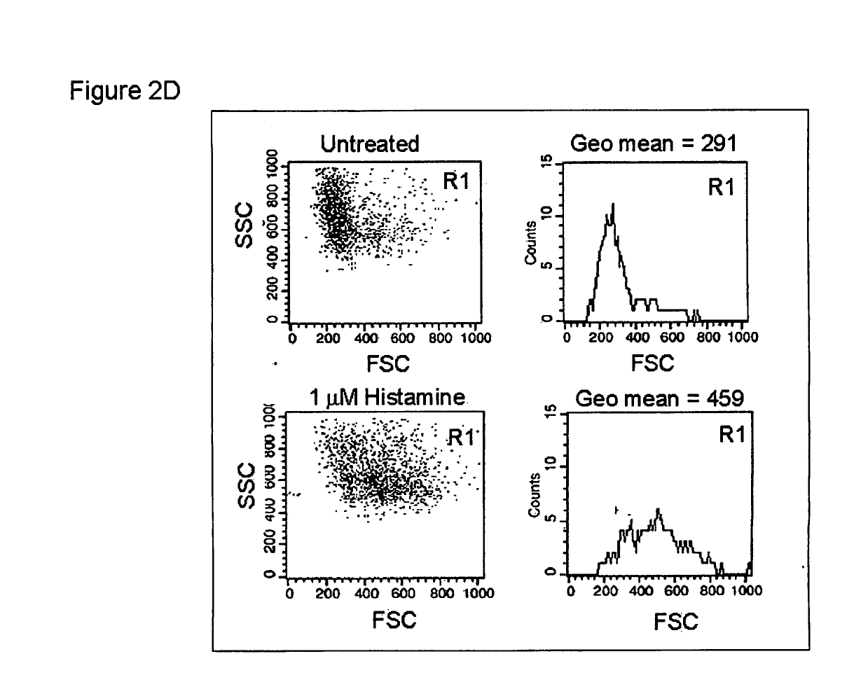

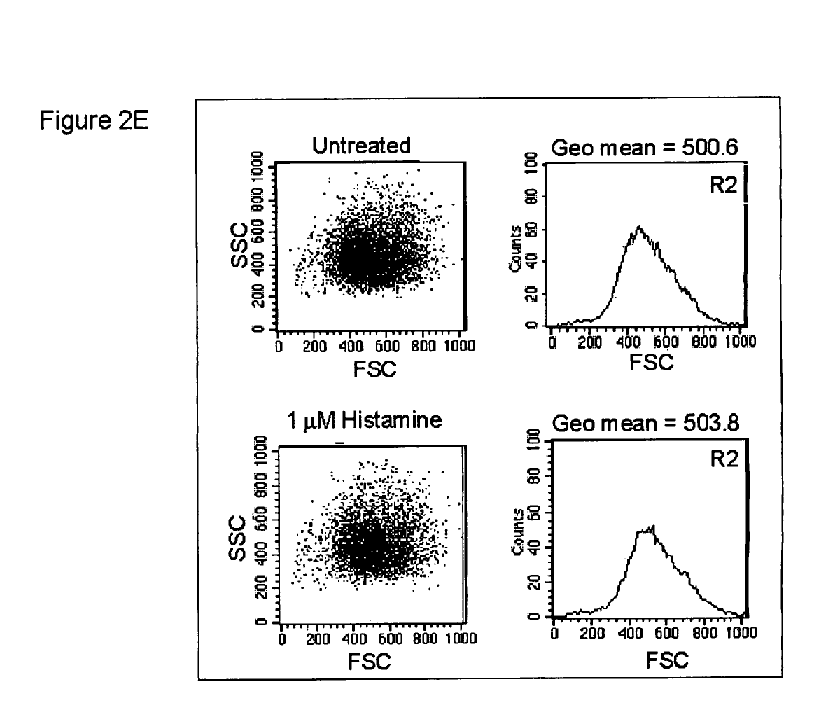

The analysis of eosinophil cell shape change by flow cytometry, known as gated autofluorescence forward scatter (GAFS) assay, allows a quantitative measurement of cell shape change induced by chemoattractants (Sabroe et al., 1999, “Differential regulation of eosinophil chemokine signaling via CCR3 and non-CCR3 pathways,” J. Immunol., 162, 2946-2955). The possible effects of histamine on the cell shape of human eosinophils were studied by flow cytometry. Polymorphonuclear leukocytes (PMNL) enriched with neutrophils and eosinophils were prepared from human blood samples and the response of these cells to histamine was studied. Eosinophils had high levels of autofluorescence and could be distinguished from neutrophils by flow cytometry (FIG. 2A). As shown in FIG. 2A, the cell population in PMNL with high levels of autofluorescence was highly enriched with CCR3⁺ eosinophils, whereas the population with low autofluorescence was composed mainly of CD16⁺ neutrophils. Histamine at 1 μM induced a significant cell shape change on eosinophils, but had no effects on neutrophils (FIGS. 2B and 2C).

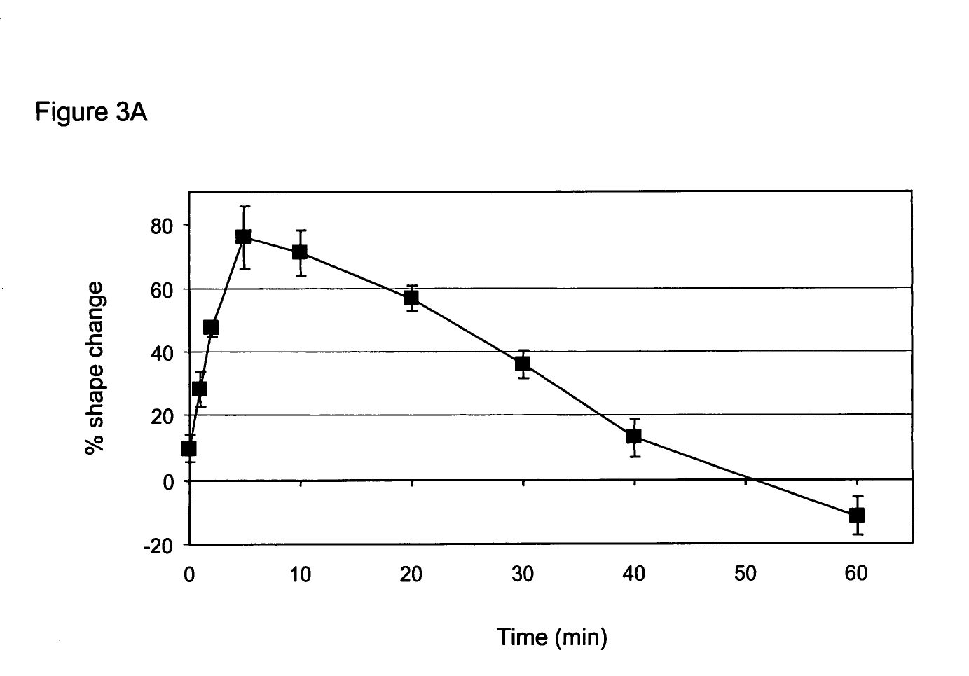

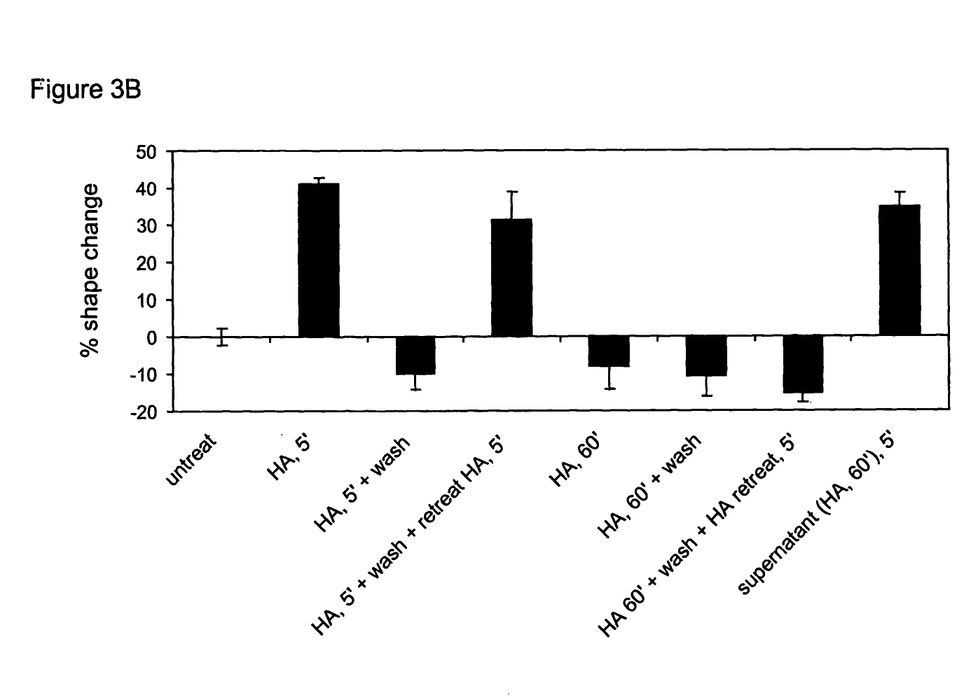

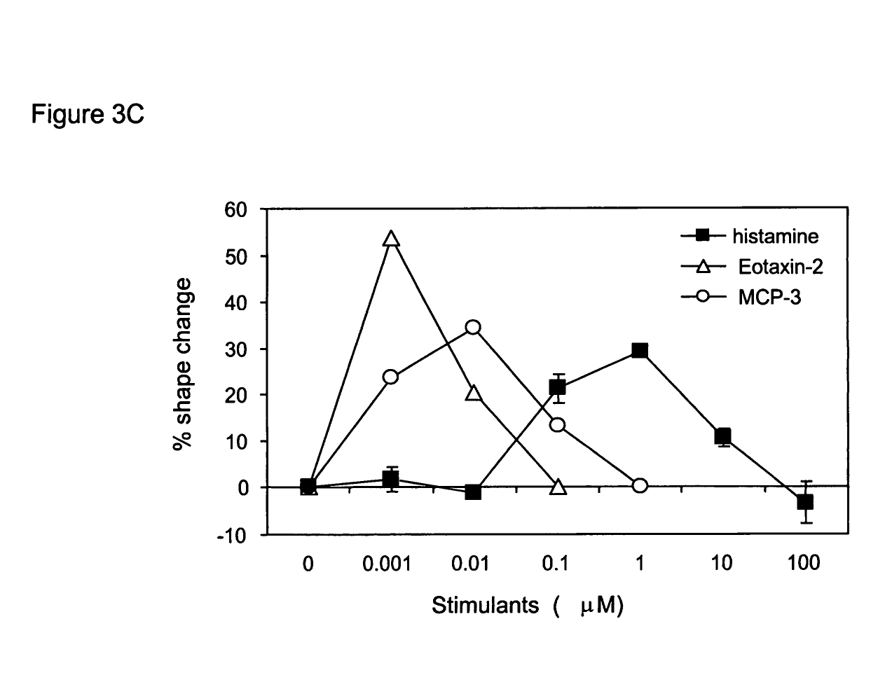

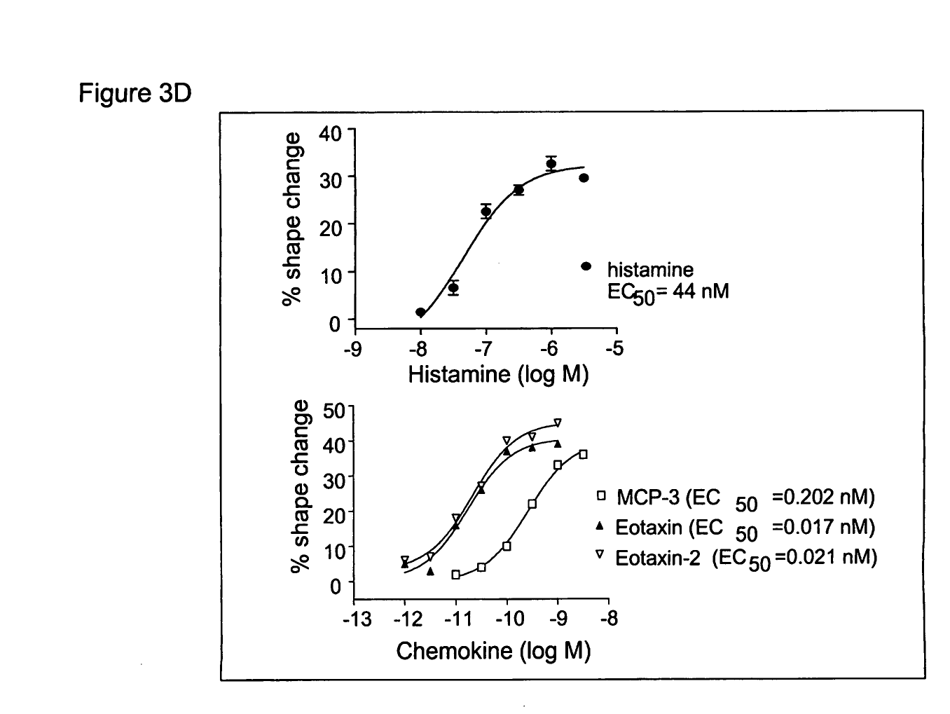

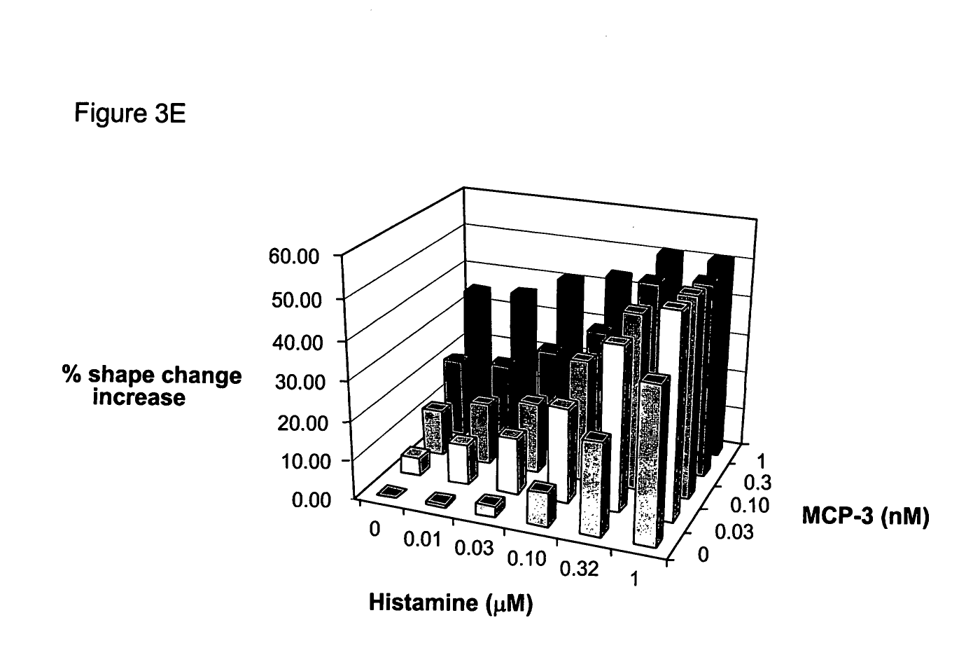

Histamine induced a rapid and transient cell shape change on eosinophils that could be detected by flow cytometry as early as 1 min after histamine treatment, with a maximal change at 5 min, and a gradual return to the original cell shape after 40 min (FIG. 3A). The disappearance of histamine effects over time was not due to the loss of histamine activity in the cell supernatants. These cell supernatants could still trigger a normal cell shape change on freshly prepared eosinophils (FIG. 3B). The eosinophil shape change was not maintained when histamine was removed. As shown in FIG. 3B, eosinophils treated with histamine for 5 min followed by washing did not retain any of the cell shape change. However, these cells were still fully capable of re-mounting shape change response when re-treated with histamine (FIG. 3B). In contrast, eosinophils after 60-min incubation with histamine not only returned to original cell shape, but also lost their response to histamine re-stimulation. These cells did not respond to histamine anymore even when they were washed free of histamine before re-stimulation (FIG. 3B).

Eosinophil shape change was induced by histamine in a concentration-dependent manner (FIG. 3C). The optimal concentration of histamine for maximal shape change on eosinophils was ˜1 μM. This shape change became less obvious when histamine concentration was higher than 1 μM, and no shape change was found at 100 μM histamine. The EC₅₀ of histamine on eosinophil shape change was 44 nM, whereas the EC₅₀ of chemokines eotaxin, eotaxin-2 and MCP-3 were 17 pM, 21 pM and 202 pM respectively (FIG. 3D).

Possible synergistic effects between histamine and chemokines in mediating eosinophil shape change were studied. A titration of both histamine and chemokine MCP-3 and their combined effects on eosinophil shape change were monitored (FIG. 3E). Partial additive effects were shown when either histamine or MCP-3 was at sub-optimal concentration range. This effect was histamine or MCP-3 concentration dependent and no further enhancement was found when they reached their maximal effective doses. Similar additive effects were also observed when histamine was combined with eotaxin-2 in eosinophil shape change studies.

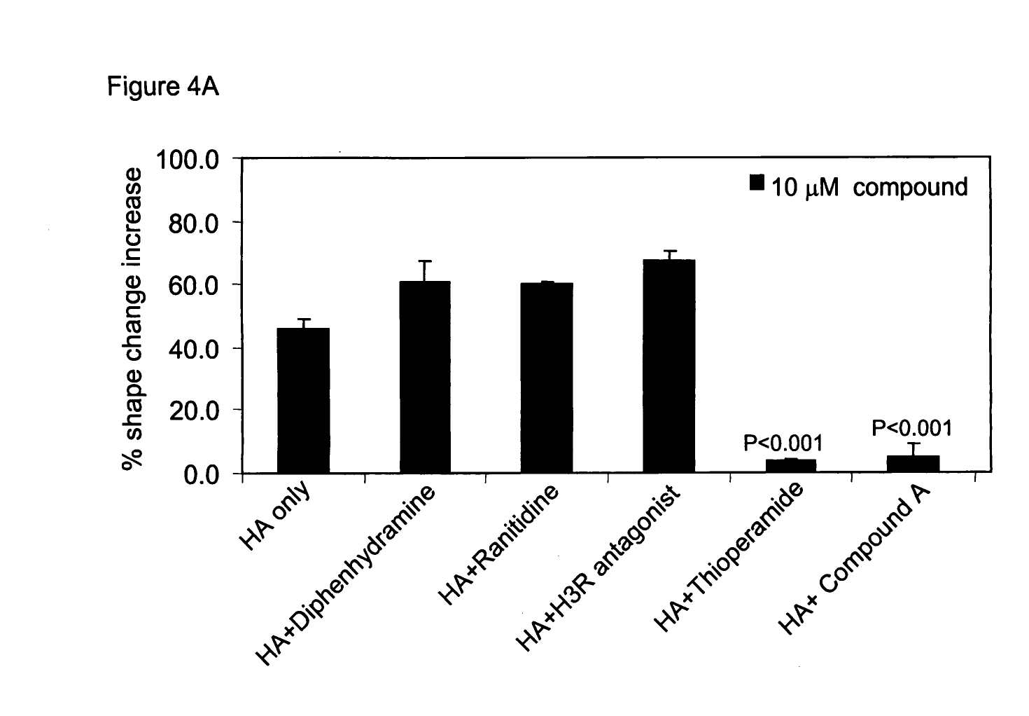

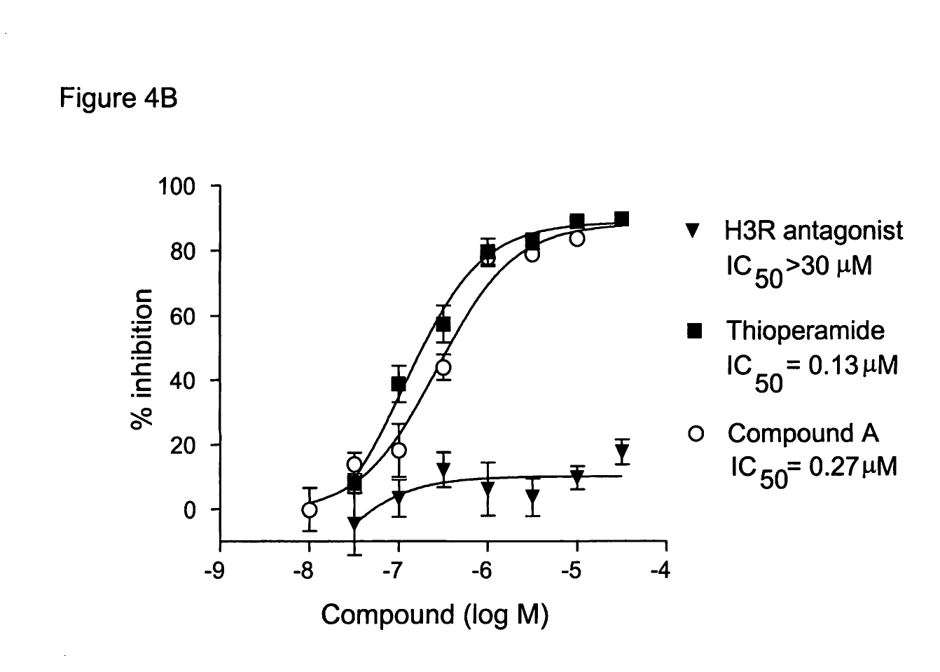

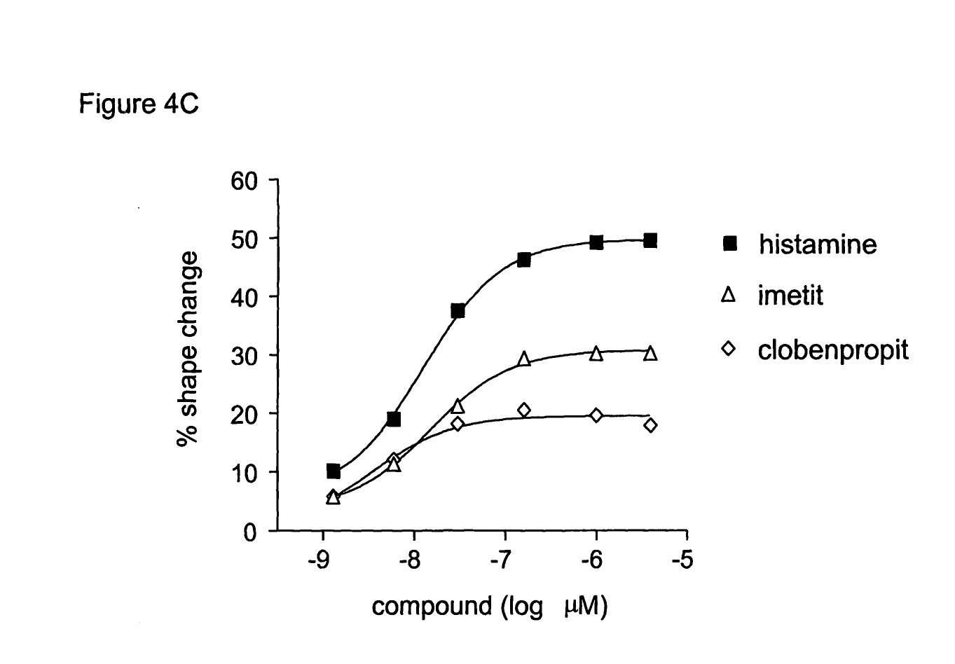

Specific histamine receptor ligands were used to determine the histamine receptor responsible for eosinophil shape change. The H₄ receptor-specific antagonist Compound A and the H₃/H₄ receptor antagonist thioperamide at 10 μM blocked the histamine-induced eosinophil shape change completely (FIG. 4A). In contrast, the H₃ receptor antagonist Compound B, the H₁ receptor antagonist diphenhydramine, and the H₂ receptor antagonist ranitidine did not show any inhibitory effects (FIG. 4A). The IC₅₀ of Compound A and thioperamide in blocking 1 μM histamine-induced eosinophil shape change was 270 nM and 128 nM, respectively, whereas the H₃ receptor specific antagonist was ineffective up to 30 μM (FIG. 4B). The H₃/H₄ receptor agonist imetit and the H₄ receptor-specific agonist clobenpropit could mimic histamine effect in triggering a partial shape change in eosinophils (FIG. 4C). Thus, histamine-induced eosinophil shape change appeared to be mediated by the H₄ receptor.

### Results: Histamine Up-Regulates Cell Surface Adhesion Molecules through H₄ Receptors.

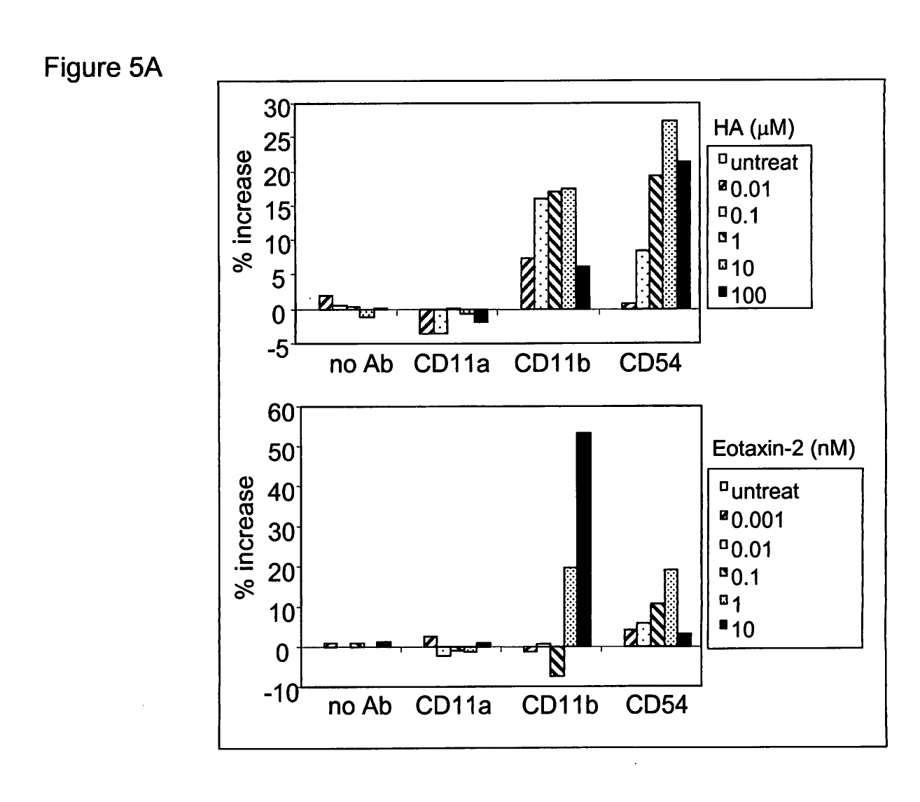

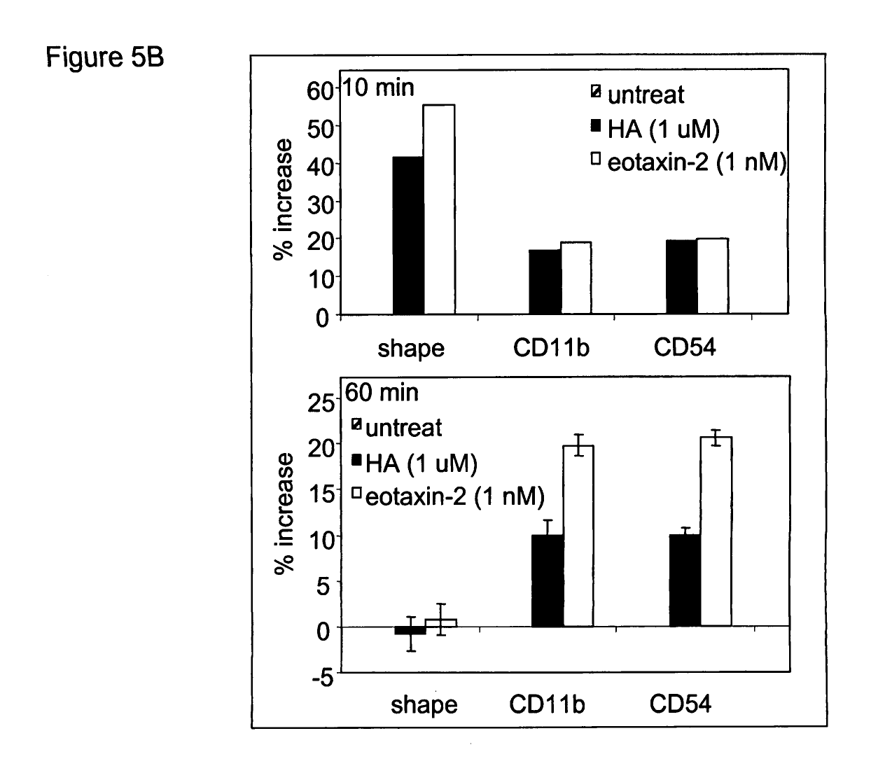

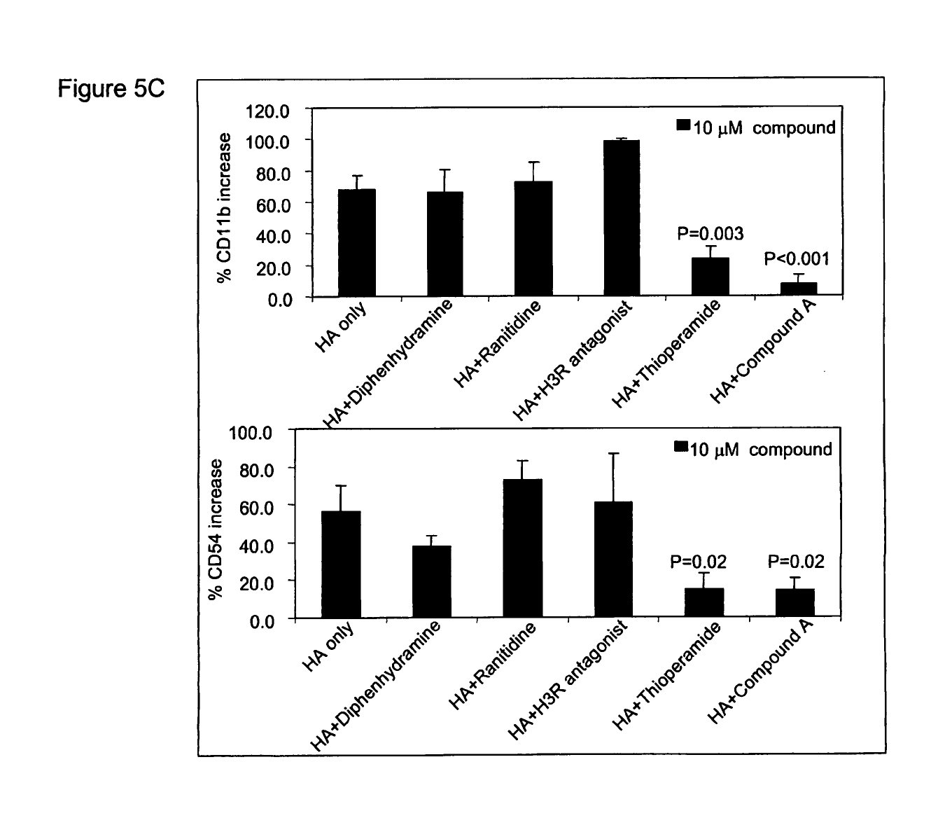

The effect of histamine on adhesion molecule expression on eosinophil cell surface was studied in flow cytometry using specific antibodies. Cell surface expression of CD11b/CD18 (Mac-1) and CD54 (ICAM-1) on eosinophils was induced by histamine in a concentration-dependent manner (FIG. 5A). The optimal histamine concentration for maximal up-regulation of CD11b/CD18 and CD54 was 10 μM with 10-minute stimulation. Interestingly, the expression of CD11a/CD18 (LFA-1), another member of the β2-intergrin as CD11b/CD18, was not induced by histamine (FIG. 5A). The increase in cell surface expression of adhesion molecules was observed at 10 min of histamine treatment (FIGS. 5A and 5B). Unlike the transient shape change response, the increase in cell surface adhesion molecules was maintained after 60 minutes incubation with histamine (FIG. 5B).

The histamine receptor responsible for adhesion molecule up-regulation on eosinophils was investigated using different specific histamine receptor antagonists. As shown in FIG. 5C, 10 μM of either Compound A or thioperamide abolished the up-regulation of CD11b/CD18 and CD54 expression. In contrast, diphenhydramine, ranitidine, and the H₃ receptor antagonist Compound B were all ineffective in blocking the up-regulation of adhesion molecules on eosinophils.

### Results: Histamine Mediates Eosinophil Chemotaxis through H₄ Receptors.

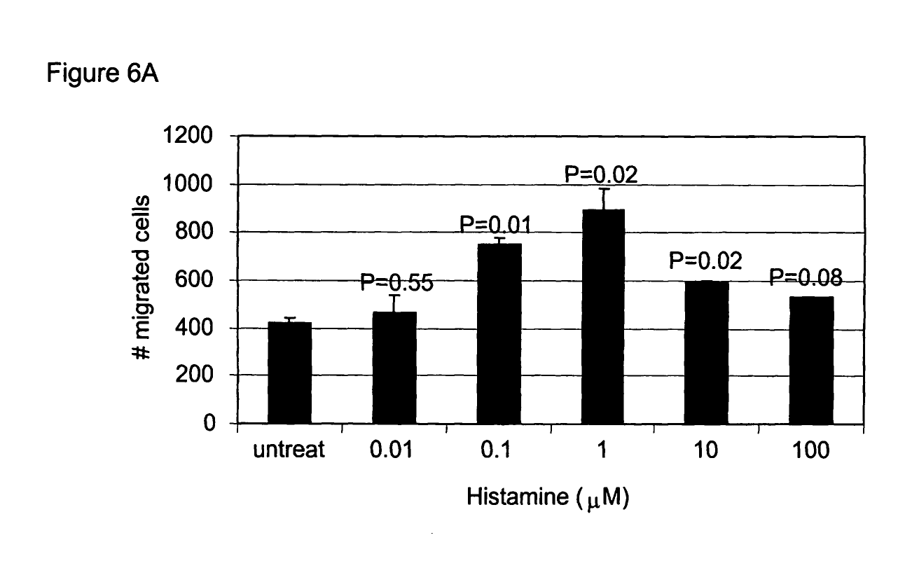

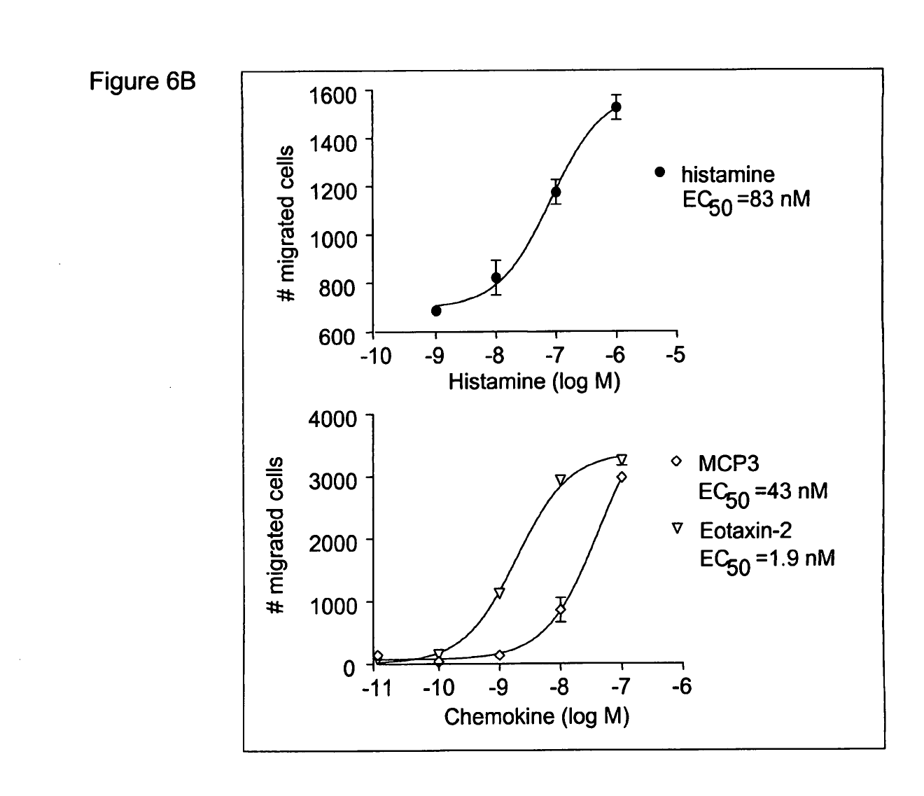

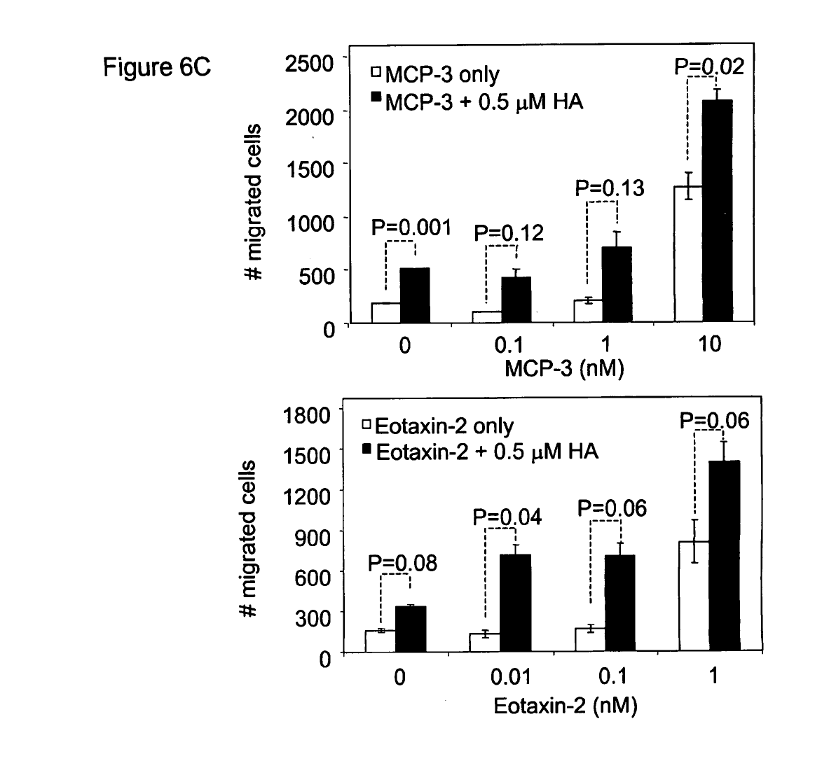

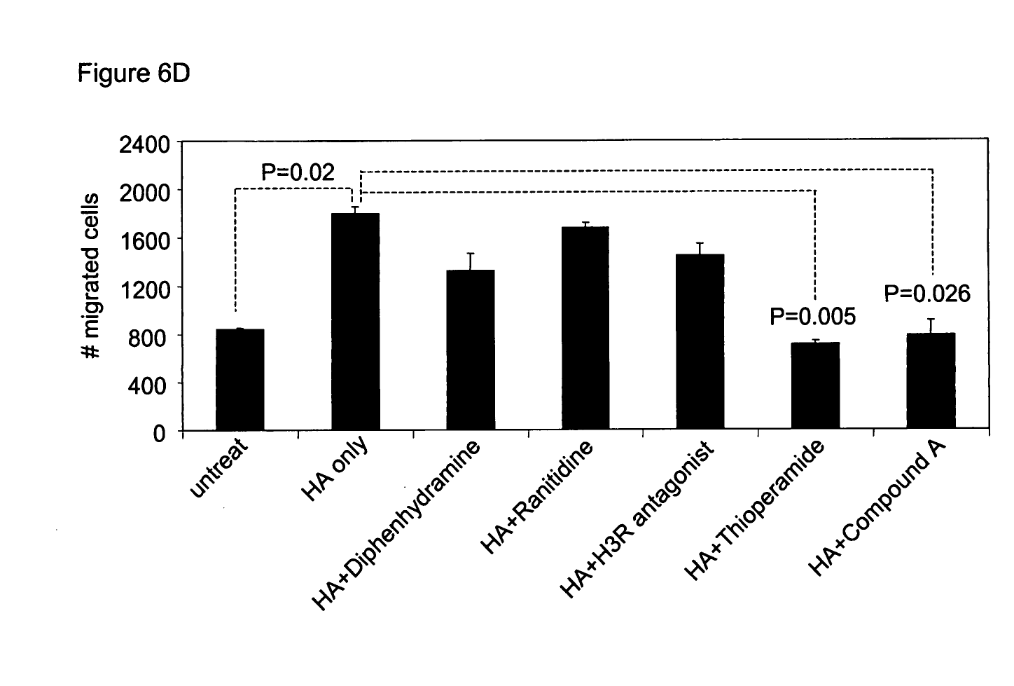

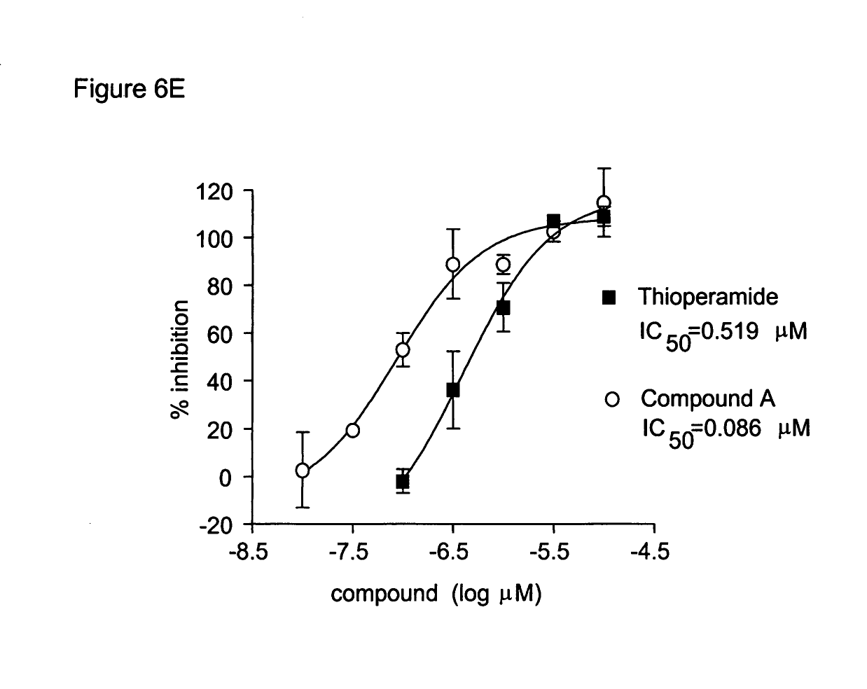

The chemotactic effects of histamine on eosinophils were investigated using purified human blood eosinophils. In vitro chemotaxis studies were performed in a Transwell system. Histamine induced eosinophil migration in a concentration-dependent manner with an EC₅₀ of 83 nM (FIGS. 6A and 6B). A maximal chemotactic effect on eosinophils was achieved at 1 μM histamine (FIG. 6A). Chemokines eotaxin-2 and MCP-3 were used in same assays for comparison. The EC₅₀ of chemokines eotaxin-2 and MCP-3 on eosinophil chemotaxis was 1.9 nM and 43 nM, respectively (FIG. 6B). The histamine effect on eosinophils was chemotactic but not chemokinetic, since disruption of the histamine concentration gradient abolished eosinophil migration completely.

Possible synergistic effects between histamine and chemokines on eosinophil chemotaxis were studied. A titration of chemokines eotaxin-2 and MCP-3 was performed in the presence or absence of a sub-optimal concentration of histamine, and the effect on eosinophil chemotaxis was studied. As shown in FIG. 6C, addition of 0.5 μM histamine enhanced the chemotaxis of eosinophils induced by eotaxin-2 and MCP-3.

Histamine receptor antagonists were used to determine the histamine receptor responsible for eosinophil chemotaxis. Thioperamide and Compound A at 10 μM inhibited completely the histamine-induced eosinophil chemotaxis, whereas the inhibitory effect of diphenhydramine, ranitidine or H₃ receptor antagonist was minimal (FIG. 6D). Both Compound A and thioperamide showed a concentration-dependent effect in blocking 1 μM histamine-induced eosinophil chemotaxis with an IC₅₀ of 86 nM and 519 nM, respectively (FIG. 6E). The results suggest that the histamine-induced eosinophil chemotaxis is mediated by the H₄ receptor.

### Discussion of Experimental Results.

As shown above, eosinophils expressed the H₄ but not the H₃ receptor. H₄ receptors were also expressed at significant levels in dendritic cells and at low levels in CD4⁺ effector T cells. In contrast to other reports (Morse et al., 2001, supra; Zhu et al., 2001, supra; Oda et al., 2000, supra), we were unable to detect H₄ receptor RNA message in neutrophils and monocytes.

The expression of H₄ receptor in eosinophils, mast cells, basophils and dendritic cells suggests a possible involvement of histamine and the H₄ receptor in allergic responses. The establishment of a typical allergic response involves two different stages: the allergen sensitization stage and the allergic reaction stage. During the allergen sensitization stage, dendritic cells acquire antigens and migrate to draining lymph nodes for T cell activation. Histamine released from mast cells may affect dendritic cell function via the H₄ receptor and influence T cell activation. At the stage of allergen challenge, exposure of mast cells to allergens leads to mast cell degranulation and the release of histamine. Histamine may enhance the accumulation of mast cells at sites of allergic reaction and recruit eosinophils as a late-phase response. It has been shown previously that histamine H₁ and H₂ receptors are expressed differentially on type I and type II helper T cells and they play a role in the modulation of T cell effector functions (Jutel et al., 2001, “Histamine regulates T-cell and antibody responses by differential expression of H₁ and H₂ receptors,” Nature, 413, 420-425). It is possible that the H₄ receptor is another histamine receptor involved in the complicated process of allergic responses.

The results of the above-described experiments show that histamine is a chemoattractant for eosinophils. Eosinophils respond to histamine with cell shape change, up-regulation of adhesion molecules on the cell surface, as well as chemotaxis. Using a H₄ receptor specific antagonist, the results demonstrate that all of these histamine effects on cosinophils were mediated by the H₄ receptor. Chemotaxis is a directional cell movement up a chemoattractant gradient and requires an establishment of cell polarity and thus a cell shape change toward a directional signal. Using the GAFS assay to measure eosinophil shape change, we demonstrated that histamine induced a rapid shape change in eosinophils in a concentration-dependent manner. Imetit is known to be an agonist for both H₃ and H₄ receptors, whereas clobenpropit behaves as an agonist for the H₄ receptor but an antagonist for the H₃ receptor (Oda et al., 2000, “Molecular cloning and characterization of a novel type of histamine receptor preferentially expressed in leukocytes,” J. Biol. Chem., 275, 36781-36786; Liu et al., 2001, “Cloning and pharmacological characterization of a fourth histamine receptor (H₄) expressed in bone marrow,” Mol. Pharmacol., 59, 420-426; Liu et al., 2001, “Comparison of human, mouse, rat, and guinea pig histamine H₄ receptors reveals substantial pharmacological species variation,” J. Pharmacol. Exp. Ther., 299, 121-130; Morse et al., 2001, “Cloning and characterization of a novel human histamine receptor,” J. Pharmacol. Exp. Ther., 296, 1058-1066 Morse et al., 2001; Zhu et al., 2001, “Cloning, expression, and pharmacological characterization of a novel human histamine receptor,” Mol. Pharmacol., 59, 434-441). In our studies, both imetit and clobenpropit worked as agonists and mimicked histamine effect in inducing eosinophil shape change. This result further confirms that the eosinophil shape change induced by histamine is mediated by the H₄ receptor.

The eosinophil shape change response to histamine was rapid but transient. A shape change was observed after 1-min incubation with histamine and a maximal response was reached at 5 min. Removal of histamine abolished the eosinophil shape change immediately. After short exposure to histamine, eosinophils were still fully capable of re-mounting a shape change response upon histamine re-stimulation. However, this eosinophil shape change response disappeared after incubation with histamine at high concentrations (>100 μM) or over a long period of time (>40 minutes). These eosinophils appeared to be desensitized and were no longer responsive to histamine upon re-stimulation. Receptor internalization has been reported to account for the desensitization of eosinophil response to eotaxin (Zimmermann et al., 2003, “Receptor internalization is required for eotaxin-induced responses in human eosinophils,” J. Allergy Clin. Immunol., 111, 97-105). Histamine has also been shown to induce internalization of H₄ receptors (Nguyen et al., 2001, “Discovery of a novel member of the histamine receptor family,” Mol. Pharmacol., 59, 427-433). It is possible that the desensitization of the eosinophil response to histamine that we observed here is also the result of H₄ receptor internalization.

Leukocyte chemoattractants are known to initiate a coordinated sequence of adhesive interactions between cells in circulating blood and in the microvascular endothelium. The phases of leukocyte migration are comprised of adhesion, spreading, diapedesis of the vessel endothelial cells, and infiltration into tissues (Springer, 1994, “Traffic signals for lymphocyte recirculation and leukocyte emigration: the multistep paradigm,” Cell, 76, 301-314). Up-regulation of adhesion molecules on the cell surface is essential for cell spreading and diapedesis in the process of cell migration. The results show that cell surface expression of CD11b/CD18 (Mac-1) and CD54 (ICAM-1) was up-regulated by histamine via the H₄ receptor. This up-regulation occurred within 10 minutes and is probably independent of protein synthesis. Unlike the transient shape change response, adhesion molecule up-regulation on the cell surface was retained for a much longer time (up to 60 min). Although CD11a/CD18 (LFA-1) expression level was not induced by histamine, change in the avidity toward its ligands such as ICAM-1, ICAM-2 and ICAM-3 cannot be excluded.

The effect of H₄ receptors in mediating eosinophil chemotaxis toward histamine was demonstrated in a Transwell in vitro system. The histamine effect on eosinophils was chemotactic but not chemokinetic. Compared to other typical eosinophil chemokines, histamine is a relatively weak chemotactic factor. The effective concentrations of histamine in triggering eosinophil shape change and chemotactic response are higher than those induced by chemokines such as eotaxin-2 and MCP-3. In addition, the histamine half-life in serum is very short (around 1 min) and its serum concentration is in the range of 1 nM in normal conditions and may only reach 10 nM in a systemic allergic response (Church et al., 1993, chapter 5.6, “Mast cell and basophil function,” in Allergy (Hogate et al., eds.), Raven Press Ltd., New York). Considering that the EC₅₀ of histamine for eosinophil chemotaxis as measured is 83 nM, it is unlikely that histamine in the blood stream is able to trigger eosinophil migration. However, the histamine concentration in tissues may reach a much higher level, in particular in area where mast cell degranulation has occurred. Histamine released in tissues may form complexes with heparin sulfate to prolong its half-life and to interact with extra-cellular matrix for the establishment of a histamine concentration gradient. It is possible that eosinophils rely on different chemotactic factors in their path of migration through different compartments of the in vivo system. Histamine may exert its direct chemotactic effect in tissues to recruit eosinophils after their exit from blood circulation. Exploring the possible cooperative effect of histamine with other chemokines in mediating eosinophil chemotactic response, while additive effects were observed, synergistic effects between histamine and chemokines were not observed.

In an allergic reaction, a large amount of histamine is released from mast cells locally at sites of allergen exposure. Eosinophil infiltration follows as a late phase response and these cells play a major role in the pathogenesis of allergy. The accumulated numbers of mast cells, basophils, and eosinophils at sites of allergic reactions often correlate with disease severity (Bousquet et al., 1990, “Eosinophilic inflammation in asthma,” N. Engl. J. Med., 323, 1033-1039; Macfarlane et al., 2000, “Basophils, eosinophils and mast cells in atopic and nonatopic asthma and in late-phase allergic reactions in the lung and skin,” J. Allergy Clin. Immunol., 105, 99-107). The results here establish a role of histamine in recruiting specific inflammatory cell types into sites of allergic response. Eosinophils are one of the key participants in chronic allergic diseases. The H₄ receptor-mediated eosinophil responses, such as cell shape change and up-regulation of adhesion molecules, can be detected in whole blood and therefore can serve as biomarkers in animal or clinical studies of H₄ receptor antagonists, prognostic or diagnostic tests for investigating diseases and medical conditions mediated through the H₄ receptor or determining whether a particular experimental or marketed drug might be useful in treating a particular patient. Such diseases and medical conditions include allergic airway diseases as well as those described in the following, the disclosures of which are incorporated by reference herein: International Publication Nos. WO 02/072548 and WO 02/056871; and U.S. Patent Application Publication Nos. US 2003-0133931 and US 2004-0058934.

Accordingly, the invention is directed to general methods for assaying whole blood as described in the above summary. In such an assay, blood samples may be advantageously used directly without the need for any dilution or other manipulation or preparation prior to the assay. The concentrations of investigational or marketed drug compounds in blood samples may be maintained and their effects in blocking, inhibiting, or modulating H₄ receptor function can be conveniently monitored ex vivo.

As a control sample, whole blood may be obtained from a source, such as an animal, e.g., the subject of an animal model, or a human, e.g., the subject of a clinical trial or a patient having a disease or medical condition believed to be mediated by histamine H₄ receptor activity. For the assay sample, the sample is obtained from an animal or human whose blood has been treated with an H₄ receptor antagonist, e.g., through in vivo dosing of the subject by administering the H₄ receptor antagonist or direct addition to the blood ex vivo. Exemplary animals include mammals such as mice, rats, guinea pigs, dogs, and sheep.

Exemplary H₄ receptor antagonists that may be assayed for their efficacy include those described in the following, the disclosures of which are incorporated by reference herein: International Publication Nos. WO 02/072548 and WO 02/056871; and U.S. Patent Application Publication Nos. US 2003-0133931 and US 2004-0058934.

Additional examples of H₄ receptor antagonists include members of the general classes based on the following headgroups or moieties:

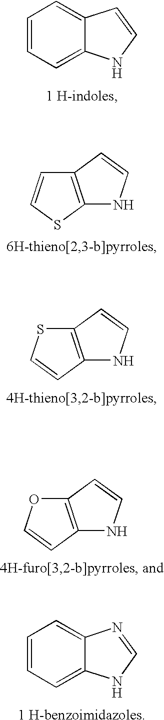

Specific examples of such H₄ receptor antagonists include the following compounds:
(5-CHLORO-7-METHYL-1H-INDOL-2-YL-(4-METHYL-PIPERAZIN-1-YL)-METHANONE;
(2-CHLORO-3-METHYL-4H-THIENO[3,2-B]PYRROL-5-YL)-(4-METHYL-PIPERAZIN-1-YL)-METHANONE;
(5-CHLORO-1H-INDOL-2-YL)-(4-METHYL-PIPERAZIN-1-YL)-METHANONE;
(2,3-DIMETHYL-4H-THIENO[3,2-B]PYRROL-5-YL)-(4-METHYL-PIPERAZIN-1-YL)-METHANONE;
(2,3-DICHLORO-4H-THIENO[3,2-B]PYRROL-5-YL)-(4-METHYL-PIPERAZIN-1-YL)-METHANONE;
(7-METHYL-1H-INDOL-2-YL)-(4-METHYL-PIPERAZIN-1-YL)-METHANONE;
(7-AMINO-1H-INDOL-2-YL)-(4-METHYL-PIPERAZIN-1-YL)METHANONE;
(5-BROMO-1H-INDOL-2-YL)-(4-METHYL-PIPERAZIN-1-YL)-METHANONE;
(5,7-DICHLORO-1H-INDOL-2-YL)-(4-METHYL-PIPERAZIN-1-YL)-METHANONE;
(5-CHLORO-1H-INDOL-2-YL)-PIPERAZIN-1-YL-METHANONE;
(2,3-DICHLORO-6H-THIENO[2,3-B]PYRROL-5-YL)-(4-METHYL-PIPERAZIN-1-YL)-METHANONE;
(5-METHYL-1H-INDOL-2-YL)-(4-METHYL-PIPERAZIN-1-YL)-METHANONE;
(4,5-DICHLORO-1H-INDOL-2-YL)-(4-METHYL-PIPERAZIN-1-YL)-METHANONE;
(4-METHYL-PIPERAZIN-1-YL)-(5-TRIFLUOROMETHYL-1H-BENZOIMIDAZOL-2-YL)-METHANONE;
(5-FLUORO-1H-INDOL-2-YL)-(4-METHYL-PIPERAZIN-1-YL)-METHANONE;
(5,7-DIFLUORO-1H-INDOL-2-YL)-(4-METHYL-PIPERAZIN-1-YL)-METHANONE;
(5-AMINO-1H-INDOL-2-YL)-(4-METHYL-PIPERAZIN-1-YL)-METHANONE;
(5-HYDROXY-1H-INDOL-2-YL)-(4-METHYL-PIPERAZIN-1-YL)-METHANONE;
(4-METHYL-PIPERAZIN-1-YL)-(3-METHYL-4H-THIENO[3,2-B]PYRROL-5-YL)-METHANONE;
(7-CHLORO-1H-INDOL-2-YL)-(4-METHYL-PIPERAZIN-1-YL)-METHANONE;
(5-CHLORO-1H-INDOL-2-YL)-[4-(2-HYDROXY-ETHYL)-PIPERAZIN-1-YL]-METHANONE;
(2-CHLORO-6H-THIENO[2,3-B]PYRROL-5-YL)-(4-METHYL-PIPERAZIN-1-YL)-METHANONE;
(5-CHLORO-1H-BENZOIMIDAZOL-2-YL)-(4-METHYL-PIPERAZIN-1-YL)-METHANONE;
(5-FLUORO-1H-BENZOIMIDAZOL-2-YL)-(4-METHYL-PIPERAZIN-1-YL)-METHANONE;
(5-CHLORO-1H-INDOL-2-YL)-(3,4-DIMETHYL-PIPERAZIN-1-YL)-METHANONE;
(5,6-DIFLUORO-1H-BENZOIMIDAZOL-2-YL)-(4-METHYL-PIPERAZIN-1-YL)-METHANONE;
(5,7-DIMETHYL-1H-INDOL-2-YL-(4-METHYL-PIPERAZIN-1-YL)-METHANONE;
(2-CHLORO-3-METHYL-4H-THIENO[3,2-B]PYRROL-5-YL)-PIPERAZIN-1-YL-METHANONE;
(4-METHYL-1H-BENZOIMIDAZOL-2-YL)-(4-METHYL-PIPERAZIN-1-YL)-METHANONE;
(1H-BENZOIMIDAZOL-2-YL)-(4-METHYL-PIPERAZIN-1-YL)-METHANONE;
(6-HYDROXY-1-INDOL-2-YL)-(4-METHYL-PIPERAZIN-1-YL)-METHANONE;
(5-CHLORO-1H-INDOL-2-YL)-((R)-3-METHYL-PIPERAZIN-1-YL)-METHANONE;
(5-CHLORO-1H-INDOL-2-YL)-((S)-3-METHYL-PIPERAZIN-1-YL)-METHANONE;
(4-BROMO-1H-INDOL-2-YL)-(4-METHYL-PIPERAZIN-1-YL)-METHANONE;
(2-CHLORO-4H-THIENO[3,2-B]PYRROL-5-YL)-(4-METHYL-PIPERAZIN-1-YL)-METHANONE;
(5-CHLORO-1H-INDOL-2-YL)-(3-METHYL-PIPERAZIN-1-YL)-METHANONE;
(5-FLUORO-1H-BENZOIMIDAZOL-2-YL)-PIPERAZIN-1-YL-METHANONE;
(7-AMINO-1H-INDOL-2-YL)-PIPERAZIN-1-YL-METHANONE;
(4-METHYL-PIPERAZIN-1-YL)-(5-NITRO-1H-INDOL-2-YL)-METHANONE;
(7-HYDROXY-1H-INDOL-2-YL)-(4-METHYL-PIPERAZIN-1-YL)-METHANONE;
(6-CHLORO-5-FLUORO-1H-BENZOIMIDAZOL-2-YL)-(4-METHYL-PIPERAZIN-1-YL)-METHANONE;
(7-BROMO-1H-INDOL-2-YL)-(4-METHYL-PIPERAZIN-1-YL)-METHANONE;
(2-CHLORO-6H-THIENO[2,3-B]PYRROL-5-YL)-PIPERAZIN-1-YL-METHANONE;
(3-BROMO-4H-THIENO[3,2-B]PYRROL-5-YL)-(4-METHYL-PIPERAZIN-1-YL)-METHANONE;
(3-METHYL-4H-THIENO[3,2-B]PYRROL-5-YL)-PIPERAZIN-1-YL-METHANONE;
(4-METHYL-PIPERAZIN-1-YL)-(6H-THIENO[2,3-B]PYRROL-5-YL)-METHANONE;
(5-CHLORO-1H-BENZOIMIDAZOL-2-YL)-PIPERAZIN-1-YL-METHANONE;
1H-BENZOIMIDAZOLE-2-CARBOXYLIC ACID (8-METHYL-8-AZA-BICYCLO[3.2.1]OCT-3-YL)-AMIDE;
(5-BROMO-BENZOFURAN-2-YL)-(4-METHYL-PIPERAZIN-1-YL)-METHANONE;
(1H-INDOL-2-YL)-3-METHYL-PIPERAZIN-1-YL)-METHANONE;
(1H-INDOL-2-YL)-4-METHYL-PIPERAZIN-1-YL)-METHANONE;
(6-CHLORO-1H-INDOL-2-YL)-(4-METHYL-PIPERAZIN-1-YL)-METHANONE;
(4-METHYL-PIPERAZIN-1-YL)-(4H-THIENO[3,2-B]PYRROL-5-YL)-METHANONE;
(1H-INDOL-2-YL)-(4-METHYL-PIPERAZIN-1-YL)-METHANETHIONE;
(4-METHYL-1H-BENZOIMIDAZOL-2-YL)-PIPERAZIN-1-YL-METHANONE;
(2,3-DIMETHYL-4H-FURO[3,2-B]PYRROL-5-YL)-(4-METHYL-PIPERAZIN-1-YL)-METHANONE;
[5-(3-METHOXY-PHENYL)-1H-INDOL-2-YL]-(4-METHYL-PIPERAZIN-1-YL)-METHANONE;
(4-METHYL-1H-BENZOIMIDAZOL-2-YL)-(3-METHYL-PIPERAZIN-1-YL)-METHANONE;
(3-METHYL-PIPERAZIN-1-YL)-(3-METHYL-4H-THIENO[3,2-B]PYRROL-5-YL)-METHANONE;
(2-CHLORO-6H-THIENO[2,3-B]PYRROL-5-YL)-(HEXAHYDRO-PYRROLO[1,2-A]PYRAZIN-2-YL)-METHANONE;
(6-BROMO-1H-INDOL-2-YL)-(4-METHYL-PIPERAZIN-1-YL)-METHANONE;
5-METHYL-1H-BENZOIMIDAZOLE-2-CARBOXYLIC ACID (8-METHYL-8-AZA-BICYCLO[3.2.1]OCT-3-YL)-AMIDE; and
(3-BROMO-4H-THIENO[3,2-B]PYRROL-5-YL)-(3-METHYL-PIPERAZIN-1-YL)-METHANONE.

Other suitable antagonists may be selected from other compounds known in the art or that become available.

To the blood sample to be assayed is added histamine (4-(2-amino-ethyl)imidazole, which may be used in a suitable form, e.g., as a hydrochloride or phosphate) or a specific histamine H₄ receptor agonist, such as imetit or clobenpropit, as the assay reagent. Other suitable agonist compounds may be selected from those known in the art or that become available.

After addition of the assay reagent, the assay sample is reacted at a suitable incubation temperature and time, such as from about room temperature to about 40° C. and a time of from about 1 to 60 minutes. For example, incubation may be carried out at a temperature of about 37° C. and a time of about 10 minutes. Following incubation, the reaction is quenched, e.g., with an ice-cold water bath. Following termination of the reaction, the sample is fixed with a suitable fixative, such as 2% paraformaldehyde in PBS.

To facilitate detection of eosinophil shape change, the red cells of the assay sample are lysed, e.g., with a hypotonic lysing solution such as Qiagen or Optilyse. To analyze cell shape change, an autofluorescence analyzer or a flow cytometer, such as a FACScan flow cytometer, may be employed.

Alternatively, other histamine H₄ receptor-mediated effects may be analyzed. For example, intracellular calcium flux, adhesion molecule up-regulation, or cytoskeletal changes may be detected.

Illustrative embodiments of assays of the invention are described in the following examples.

### EXAMPLES

### Examples 1A and 1B

### Measurement of Eosinophil Shape Change using Flow Cytometry

### Example 1A

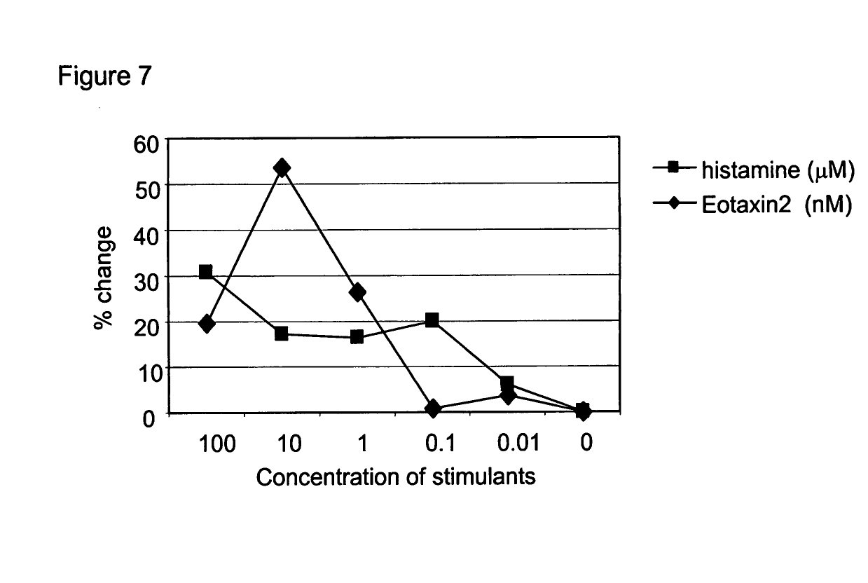

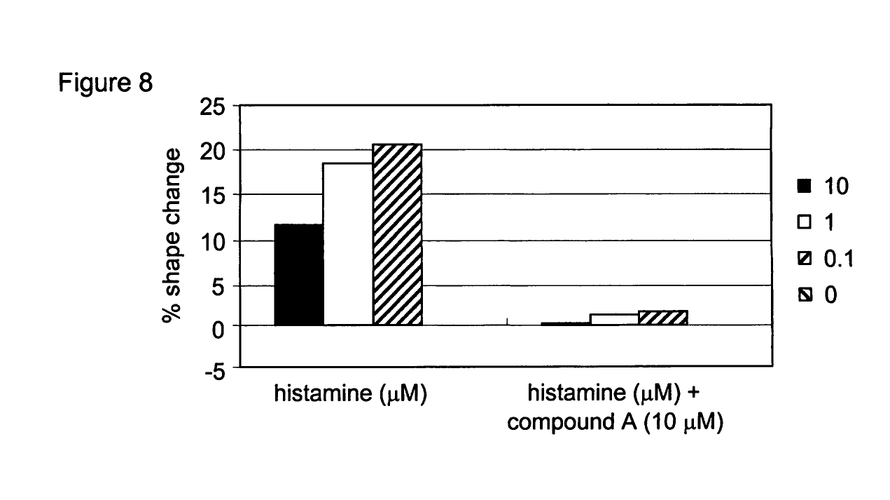

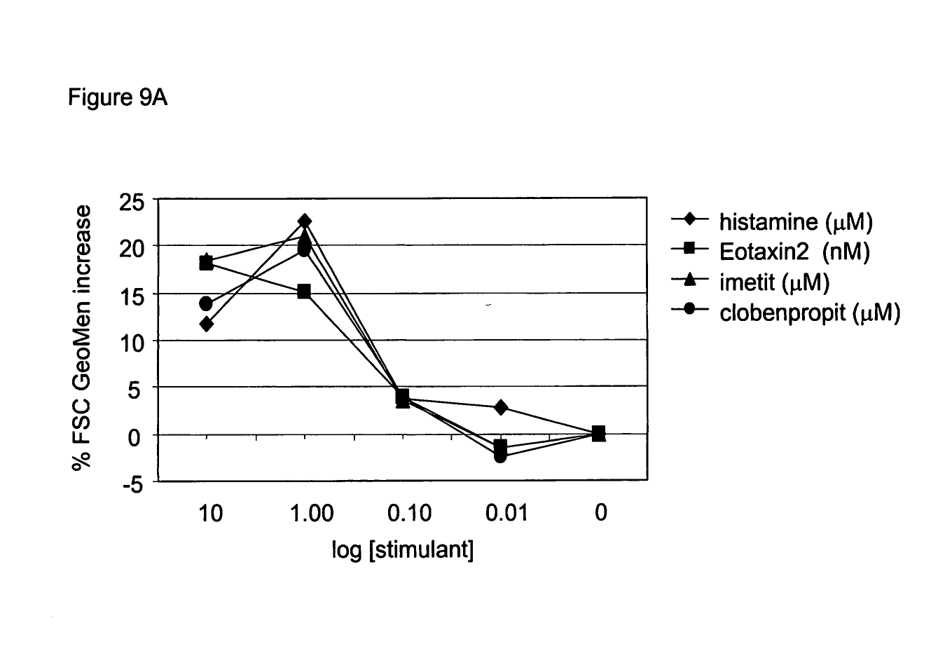

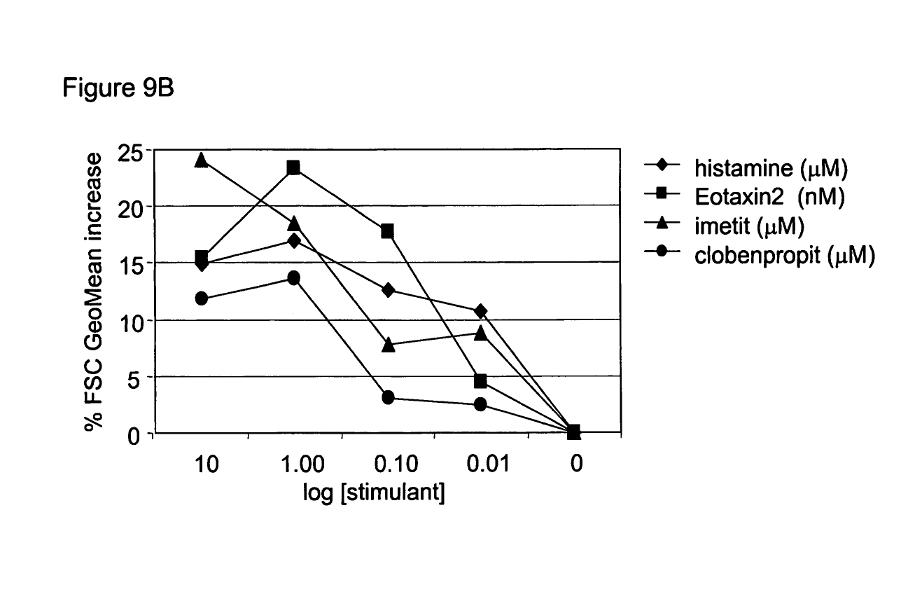

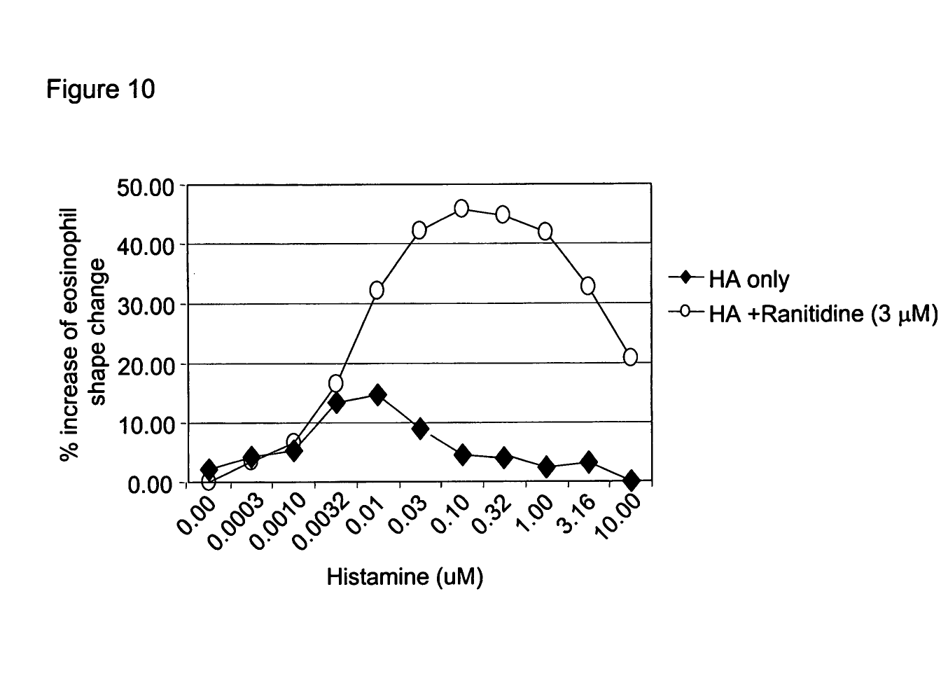

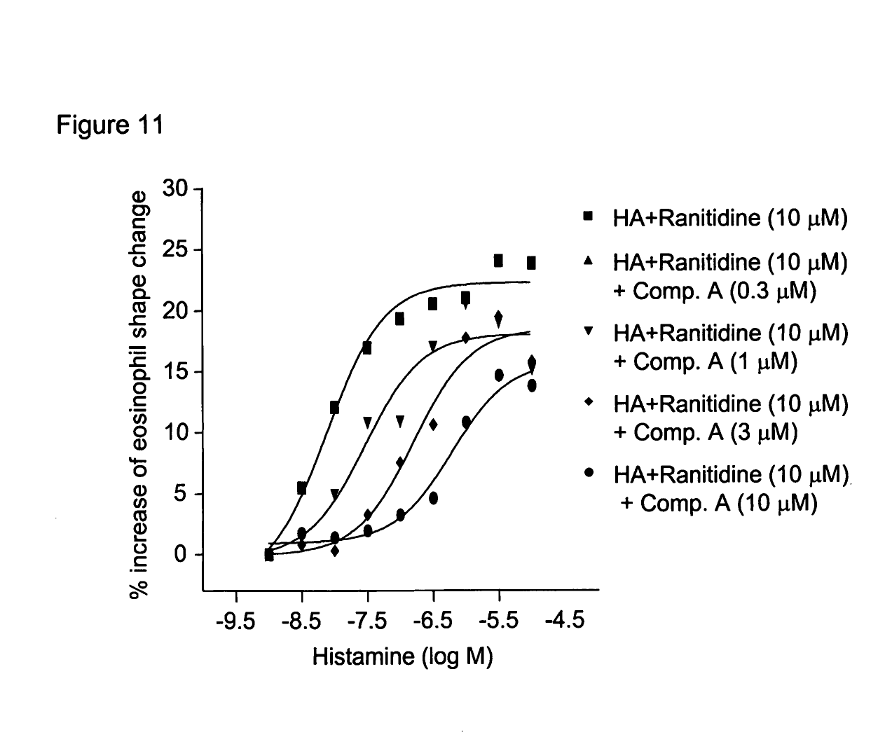

Starting materials were obtained as described in the experiments above. Human whole blood samples were used to study eosinophil shape change response. Human blood samples collected from healthy donors were treated with 3.8% tri-sodium citrate as anti-coagulator (or other anti-coagulators) and centrifuges at 300 rpm. The samples were used for experiments within 1 hour. Aliquots of 80 μl whole blood were pretreated with histamine receptor analogues (such as H₁ receptor antagonist diphenhydramine, H₂ antagonist ranitidine, H₄ receptor antagonists thioperamide and Compound A, and H₃ receptor antagonist Compound B) for 10 minutes before addition of histamine or chemokines in 1.2-ml polypropylene cluster tubes (Costar, Cambridge, Mass.) in a final volume of 100 μl. The tubes were placed in a 37° C. water bath for 10 min, after which they were transferred to an ice-water bath, and 250 μl of ice-cold fixative (2% paraformaldehyde in PBS or other fixatives) was added to terminate the reaction and to maintain the cell shape change. Red cells were lysed with 3 ml Qiagen lysis solution (Qiagen, Valencia, Calif.) for 10 minutes at 4° C. Leukocytes were recovered by centrifugation at 2000 rpm for 5 minutes, washed once with either PBS containing 10 mM Ca²⁺ and Mg²⁺, 10 mM HEPES, 10 mM glucose, and 0.1% BSA (for FIGS. 10 and 11) or PBS containing 0.5% BSA and 2 mM EDTA (for other figures), and then resuspended in the same buffer. The cell shape change was analyzed on a FACScan flow cytometer (Becton Dickinson, Mountain View, Calif.). Eosinophils were gated based on their high autofluorescence relative to neutrophils and shape change was monitored in forward scatter signals. Data were acquired for 1500 high-fluorescence eosinophil events. The results are expressed as the percentage increase in FSC compared to unstimulated cells. Results obtained are provided in the Tables I and II, below, which also correspond to FIG. 7 and FIG. 8, respectively.

TABLE I

| | histamine | histamine | Eotaxin2 |
|---|---|---|---|
| | conc. | (uM) | (nM) |
| GeoMean | 100 | 295.8 | 269.3 |
| | 10 | 265.0 | 346.0 |
| | 1 | 263.3 | 284.7 |
| | 0.1 | 271.3 | 227.7 |
| | 0.01 | 239.8 | 233.4 |
| | 0 | 226.1 | 225.5 |

| | | | histamine (uM) + |
|---|---|---|---|
| | histamine | histamine | Compound A |
| | conc. | (uM) | (10 uM) |
| GeoMean | 10 | 303.3 | 267.3 |
| | 1 | 321.9 | 270.9 |
| | 0.1 | 327.7 | 272.1 |
| | 0.00 | 272.8 | 268.1 |

TABLE II

| | histamine | histamine | Eotaxin2 |
|---|---|---|---|
| | conc. | (uM) | (nM) |
| % change | 100 | 30.8 | 19.4 |
| | 10 | 17.2 | 53.4 |
| | 1 | 16.4 | 26.3 |
| | 0.1 | 20.0 | 1.0 |
| | 0.01 | 6.1 | 3.5 |
| | 0 | 0.0 | 0.0 |

| | | | histamine (uM) + |
|---|---|---|---|
| | histamine | histamine | compound A |
| | conc. | (uM) | (10 uM) |
| % change | 10 | 11.1 | −0.3 |
| | 1 | 18.0 | 1.1 |
| | 0.1 | 20.1 | 1.5 |
| | 0 | 0.0 | 0.0 |

Eosinophils could be distinguished from other leukocytes in blood samples by their distinct high levels of autofluorescence that allowed gating of these cells for further analysis. As reflected in FIG. 7, the degree of histamine-induced eosinophil shape change was dependent on histamine concentrations. A significant eosinophil shape change was detected with ˜0.1 μM histamine. The chemokine eotaxin-2 was used as a positive control in the assay. Eosinophil shape changes induced by histamine from 0.1 to 10 μM were blocked by Compound A (FIG. 8).

### Example 1B

In this example, starting materials were obtained as described in Example 1A. Human whole blood samples were used to study eosinophil shape change response. Human blood samples collected from healthy donors were treated with 3.8% tri-sodium citrate as anti-coagulator (or other anti-coagulators) and centrifuges at 300 rpm. The samples were used for experiments within 1 hour. Aliquots of 80 μl whole blood were pretreated with histamine receptor analogues (such as H₁ receptor antagonist diphenhydramine, H₂ antagonist ranitidine, H₄ receptor antagonists thioperamide and Compound A, and H₃ receptor antagonist Compound B) for 5 minutes before addition of histamine or chemokines in 1.2-ml polypropylene cluster tubes (Costar, Cambridge, Mass.) in a final volume of 100 μl. The tubes were placed in a 37° C. water bath for 5 min, after which they were transferred to an ice-water bath, and 150 μl of ice-cold fixative (2% paraformaldehyde in PBS or other fixatives) was added to terminate the reaction and to maintain the cell shape change. Red cells were lysed with 3 ml Qiagen lysis solution (Qiagen, Valencia, Calif.) for 10 minutes at 4° C. Leukocytes were recovered by centrifugation at 2000 rpm for 5 minutes, washed once with either PBS containing 10 mM Ca²⁺ and Mg²⁺, 10 mM HEPES, 10 mM glucose, and 0.1% BSA (for FIGS. 10 and 11) or PBS containing 0.5% BSA and 2 mM EDTA (for other figures), and then resuspended in the same buffer. The cell shape change was analyzed on a FACScan flow cytometer (Becton Dickinson, Mountain View, Calif.). Eosinophils were gated based on their high autofluorescence relative to neutrophils and shape change was monitored in forward scatter signals. Data were acquired for 1500 high-fluorescence eosinophil events. Results are expressed as the percentage increase in FSC compared to unstimulated cells.

As reflected in FIG. 10, the eosinophil shape change observed upon treatment with histamine was increased by the addition of an H₂ receptor antagonist, ranitidine, thus resolving the H₄-receptor dependent shape-change response. This H₄ receptor-dependent effect was antagonized by subsequent treatment with Compound A in a dose-dependant manner (FIG. 11).

### Example 2

### Detection of Histamine-Mediated Adhesion Molecule Up-Regulation

This example describes how human whole blood may be used to study cell surface expression of adhesion molecules. Human blood samples collected from healthy donors are treated with 3.8% tri-sodium citrate as anti-coagulator and whole blood samples are used for experiments within 1 hour. Aliquots of 80 μl whole blood are pretreated with histamine receptor analogues (diphenhydramine, ranitidine, thioperamide, Compound A, and H₃ receptor antagonist Compound B) for 10 minutes before addition of histamine in 1.2-ml polypropylene cluster tubes (Costar, Cambridge, Mass.) in a final volume of 100 μl. The tubes are placed in a water bath at a temperature and time suitable for incubation, such as about 37° C. for about 10 min (if a lower temperature such as room temperature is used, the incubation time will be longer than 10 minutes as appropriate), after which they are transferred to an ice-water bath, and 250 μl of ice-cold fixative (2% paraformaldehyde in PBS) is added to terminate the reaction and to maintain the up-regulation of adhesion molecules. Samples are then incubated on ice for 30 min with a saturating concentration of either fluorescent dye-labeled (e.g., FITC- or phycoerytherine-conjugated) anti-CD11b or anti-CD54 antibodies, washed, and then analyzed by flow cytometry.

### Example 3

### Detection of Histamine-Mediated Calcium Flux

This example illustrates how the intracellular calcium flux response in eosinophils in human whole blood samples, induced by histamine binding to the H₄ receptor, may be measured by flow cytometry. Blood cells are loaded with fluo-3 acetoxymethyl ester (2 μM) or another suitable fluorescent dye in the presence of probenecid (2.51 μM) and Pluronic F-127 (0.02%) for 20 min, and equilibrated with calcium (1.8 μM) and magnesium (1 μM) for 5 min. Whole blood samples are pretreated with histamine receptor analogues (diphenhydramine, ranitidine, thioperamide, Compound A, and Compound B) for 10 minutes. Immediately after addition of histamine in blood samples, the levels of intracellular free calcium are monitored as changes in fluorescence by flow cytometry.

### Example 4

### Detection of Histamine-Mediated Cytoskeletal Changes in Whole Blood Samples

Histamine-induced cell shape change in eosinophils is the result of cytoskeletal rearrangement due to actin polymerization. Human blood samples collected from healthy donors are treated with 3.8% tri-sodium citrate as anti-coagulator and whole blood samples are used for experiments within 1 hour. Aliquots of whole blood are pretreated with histamine receptor analogues (diphenhydramine, ranitidine, thioperamide, Compound A, and Compound B) for 10 minutes before addition of histamine. Cytoskeletal changes are fixed with ice-cold fixative (2% paraformaldehyde in PBS) and then stained with rhodamine-linked phalloidin (1:40) for 30 min in the dark. Cells are washed several times with PBS before mounting in microslides for fluorescent microscope analysis.

### Example 5

### Histamine-Mediated Eosinophil Shape Change in Whole Blood as a Biomarker in Clinical Trials

Volunteers in clinical trials are given the H₄ receptor compounds (antagonists) according to the clinical protocol. The effect of the compounds in blocking H₄ receptor-mediated effects in vivo is tested in a whole blood shape change assay. Blood samples collected from compound-dosed individuals are treated with 3.8% tri-sodium citrate as anti-coagulator and whole blood samples are used for experiments within 1 hour. Histamine is added in aliquots of 80 μl whole blood in 1.2-ml polypropylene cluster tubes (Costar, Cambridge, Mass.) in a final volume of 100 μl. The tubes are placed in a 37° C. water bath for 10 min (or as appropriate), after which they are transferred to an ice-water bath, and 250 μl of ice-cold fixative (2% paraformaldehyde in PBS) is added to terminate the reaction and to maintain the cell shape change. Red cells are lysed with 3 ml Qiagen lysis solution (Qiagen, Valencia, Calif.) or another suitable hypotonic lysing solution for 10 minutes at 4° C. Leukocytes are recovered by centrifugation at 2000 rpm for 5 minutes, washed once with PBS containing 0.5% BSA and 2 mM EDTA, and then resuspended in the same buffer. The cell shape change is analyzed on a FACScan flow cytometer (Becton Dickinson, Mountain View, Calif.). Eosinophils are gated based on their high autofluorescence relative to neutrophils and shape change is monitored in forward scatter signals.

Although the invention has been described in detail above in reference to illustrative examples and preferred embodiments, the artisan will understand that the scope of the invention is defined not by the foregoing description, but by the appended claims as properly construed under principles of patent law.

What is claimed is:

1. A method for assaying for a histamine H₄ receptor-mediated effect, comprising:
(a) performing an investigative assay on a treated sample of whole blood by steps comprising:
treating whole blood from a source with a histamine H₄ receptor antagonist, yielding the treated sample;
adding to the treated sample an assay reagent selected from histamine and specific histamine H₄ receptor agonists, yielding an assay sample; and
analyzing the assay sample to detect the histamine H₄ receptor-mediated effect.

2. A method according to claim 1, wherein said adding step further comprises adding a histamine H₂ receptor antagonist to the treated sample.

3. A method according to claim 1, further comprising before said analyzing:
incubating the assay sample at a temperature and time to facilitate an incubation reaction; and
terminating the incubation reaction and fixing the assay sample with a fixative.

4. A method according to claim 3, wherein said analyzing comprises lysing red cells in the assay sample and detecting eosinophil shape change using flow cytometry.

5. A method according to claim 3, wherein said analyzing comprises adding a staining reagent to the assay sample and detecting adhesion molecule up-regulation using flow cytometry.

6. A method according to claim 5, wherein said staining reagent is selected from fluorescent dye-labeled and phycoerytherine-conjugated anti-CD11b and anti-CD54 antibodies.

7. A method according to claim 3, wherein said analyzing comprises monitoring levels of intracellular free calcium using flow cytometry.

8. A method according to claim 7, further comprising treating the whole blood with a fluorinating agent, and further wherein said monitoring of levels of intracellular free calcium comprises measuring changes in fluorescence.

9. A method according to claim 8, wherein said analyzing comprises staining the assay sample with a fluorescent dye.

10. A method according to claim 3, wherein said analyzing comprises observing cytoskeletal changes using a fluorescent microscope.

11. A method according to claim 10, further comprising staining the assay sample with a fluorescent dye before said analyzing.

12. A method according to claim 1, wherein the source is a human subject in a clinical study, and the method further comprises:
(b) performing a control assay on an untreated sample of whole blood from the source by steps comprising:
adding to the untreated sample the assay reagent, yielding a control sample; and
analyzing the control sample to detect the histamine-mediated effect; and
(c) comparing the results of the investigative assay on the treated sample with the results of the control assay on the control sample.

13. A method according to claim 12, wherein said adding step of the investigative assay (a) further comprises adding a histamine H₂ receptor antagonist to the treated sample, and said adding step of the control assay (b) further comprises adding said histamine H₂ receptor antagonist to the untreated sample.

14. A method according to claim 12, wherein said treating step comprises administering the histamine H₄ receptor antagonist to the human subject according to a dosing regimen, and said assay on the control sample is performed before said treating.

15. A method according to claim 14, further comprising drawing whole blood from the human subject at selected time intervals during the dosing schedule to periodically provide treated samples, and performing said investigative assay on each of the treated samples.

16. A method according to claim 1, wherein the source is a human patient undergoing diagnosis for a disease or medical condition suspected of being mediated by histamine H₄ receptor activity.

17. A method for assaying for a histamine H₄ receptor-mediated effect, comprising:
(a) performing a control assay on a sample of whole blood from a source untreated with any histamine H₄ receptor antagonist by steps comprising:
adding to the untreated sample an assay reagent selected from histamine and specific histamine H₄ receptor agonists, yielding a control sample; and
analyzing the control sample to detect the histamine H₄ receptor-mediated effect.

18. A method according to claim 17, wherein said adding step further comprises adding a histamine H₂ receptor antagonist to the untreated sample.

19. A method according to claim 17, wherein said analyzing comprises detecting eosinophil shape change, cytoskeletal change, adhesion molecule up-regulation, or calcium flux.

20. A method according to claim 19, further comprising:
(b) performing an investigative assay on a treated sample of whole blood by steps comprising:
treating whole blood from the source with a histamine H₄ receptor antagonist, yielding the treated sample;
adding to the treated sample an assay reagent selected from histamine and specific histamine H₄ receptor agonists, yielding an assay sample; and
analyzing the assay sample to detect the histamine H₄ receptor-mediated effect; and
(c) comparing results of the control assay of the control sample with results of the investigative assay of the treated sample.

21. A method according to claim 20, wherein said adding step of the control assay (a) further comprises adding a histamine H₂ receptor antagonist to the treated sample, and said adding step of the investigative assay (b) further comprises adding said histamine H₂ receptor antagonist to the untreated sample.

22. A method according to claim 20, wherein said treating of the whole blood is conducted in vivo.

23. A method according to claim 22, wherein said source is a mammal.

24. A method according to claim 22, wherein said source is a human.


SEQUENCE LISTING (4 sequences)

SEQ ID NO: 1 - DNA, 21 bp, Artificial
  Human H4 receptor 5′ forward PCR primer
atgccagatactaatagcaca

SEQ ID NO: 2 - DNA, 19 bp, Artificial
  Human H4 receptor 3′ reverse PCR primer
cagtcggtcagtatcttct

SEQ ID NO: 3 - DNA, 24 bp, Artificial
  Human H3 receptor 5′ forward PCR primer
atggagcgcgcgccgcccgacggg

SEQ ID NO: 4 - DNA, 21 bp, Artificial
  Human H3 receptor 3′ reverse PCR primer
atgaagaagaaaacatgtctg